(19) United States
(12) Patent Application Publication
(10) Pub. No.: US 2024/0033310 A1
(43) Pub. Date: Feb. 1, 2024

(54) HETEROGENEOUS MICROPARTICLES, AND SYSTEMS AND METHODS OF MAKING AND USE THEREOF

(71) Applicants: Sharon FLANK, Washington, DC (US); UNIVERSITY OF MARYLAND, COLLEGE PARK, College Park, MD (US); UNIVERSITY OF MARYLAND, BALTIMORE, Baltimore, MD (US)

(72) Inventors: Ryan SOCHOL, Silver Spring, MD (US); Michael AZARIAN, Arlington, VA (US); Ruben ACEVEDO, New Carrollton, MD (US); Sharon FLANK, Washington, DC (US); Stephen HOAG, Baltimore, MD (US)

(21) Appl. No.: 18/038,733

(22) PCT Filed: Nov. 24, 2021
(86) PCT No.: PCT/US21/60698

§ 371 Date: May 25, 2023

Related U.S. Application Data
(60) Provisional application No. 63/261,891, filed on Sep. 30, 2021.
(60) Provisional application No. 63/118,634, filed on Nov. 25, 2020.

(51) Int. Cl. A61K 35/76; A61K 8/11; B01J 13/14; A61K 9/50

(52) U.S. Cl. CPC A61K 35/76; A61K 8/11; B01J 13/14; A61K 9/5005

(57) ABSTRACT

A plurality of heterogeneous microparticles can be provided, each having a shell, a payload, and a cap. The shell can define a core and an orifice in fluid communication with the core. The payload can be disposed within the core. The cap can be coupled to the shell so as to seal the orifice. The shell and cap of the microparticles can be formed by an additive manufacturing method, for example, by patterning of photomaterials using electromagnetic radiation or a particle beam. When subjected to a triggering event, at least a portion of each orifice can be exposed from the respective cap so as to release, through the exposed orifice portion, the corresponding payload from the core of the shell. The microparticles can be used in various applications, such as controlled delivery of drugs, chemicals, or biological agents, self-healing or self-lubricating materials, and failure prevention or mitigation.

### CROSS-REFERENCE TO RELATED APPLICATIONS

The present application claims the benefit of U.S. Provisional Application No. 63/118,634, filed Nov. 25, 2020, entitled “3D Nanoprinted Microparticles and Methods of Making and Using the Same,” and U.S. Provisional Application No. 63/261,891, filed Sep. 30, 2021, entitled “3D Nanoprinted Microparticles and Methods of Making and Using the Same,” each of which is incorporated by reference herein in its entirety.

### FIELD

The present disclosure relates generally to engineered particles, and more particularly, to heterogeneous microparticles, and methods of making and using such microparticles.

### BACKGROUND

Engineered microparticles are of increasing importance to a wide range of applications. For example, microparticles can aid in the production of cellular scaffolds for wound healing, serve as vehicles for delivering drug therapies, including the potential for targeted drug therapies, and play roles in biofabrication strategies such bioassembly and three-dimensional (3D) bioprinting. Motivated by such functionalities, numerous techniques have been developed to produce microparticles with designed properties, such as batch emulsion, microfluidic emulsion, electrohydrodynamic spraying, mechanical fragmentation, and various lithographic methods (e.g., imprint lithography, photolithography, and flow lithography). However, the use of such current technologies has been limited by particle polydispersity, reproducibility, and/or difficulties in constructing sophisticated particle morphologies. Additive manufacturing (also referred to as 3D printing) has also been explored for fabrication of microscale systems. For example, direct laser writing (DLW) has been used to fabricate microparticles with tunable architectures. However, microparticles fabricated using conventional DLW techniques have been homogeneous in material and phase (i.e., consisting of 100% photocured material), and thus have limited utility.

Embodiments of the disclosed subject matter may address one or more of the above-noted problems and disadvantages, among other things.

### SUMMARY

Embodiments of the disclosed subject matter system are directed to heterogeneous microparticles that can be custom-tailored for a particular application, for example, to seal a payload within a core of the microparticle and then release the payload upon exposure to a desired trigger. Embodiments also provide systems and methods for fabricating heterogeneous microparticles using additive manufacturing techniques, and systems and methods for use of heterogeneous microparticles. According to some embodiments, the precise and individualized control over the three-dimensional (3D) microarchitecture of the microparticles afforded by the additive manufacturing techniques, coupled with the ability of the fabricated microparticles to store custom payloads that can be controllably released, can offer unique and novel capabilities for various applications, such as but not limited to timed, targeted, and/or regulated therapeutic/chemical delivery, particle-specific identification, cell/tissue engineering, diagnostics, lubrication, structural self-repair, deterrence or intrusion detection, failure detection or mitigation, and fire prevention.

In one or more embodiments, a method can comprise providing a plurality of heterogeneous microparticles. Each heterogeneous microparticle can comprise a shell, a payload, and a cap. The shell can define a core and an orifice in fluid communication with the core. The payload can be disposed within the core, and the cap can be coupled to the shell so as to seal the orifice. The cap and the shell can each comprise a respective cured photomaterial. The method can further comprise subjecting the plurality of heterogeneous microparticles to a triggering event such that at least a portion of each orifice is exposed from the respective cap so as to release, through the exposed orifice portion, the corresponding payload from the core of the shell.

In one or more embodiments, a method can comprise providing a first curable photomaterial, and forming at least part of a shell by patterning the first curable photomaterial using electromagnetic radiation and/or a particle beam. The method can further comprise loading one or more liquids into a core of the shell to form a payload. The method can also comprise providing a second curable photomaterial over the shell, and forming at least part of a cap by patterning the second curable photomaterial using electromagnetic radiation and/or a particle beam.

In one or more embodiments, a heterogeneous microparticle can comprise a shell, a payload, and a cap. The shell can comprise and/or define a core and an orifice in fluid communication with the core. The payload can be disposed within the core. The cap can be coupled to the shell so as to seal the orifice. The cap and the shell can each comprise a respective cured photomaterial. In some embodiments, a group of microparticles is provided, with the cap, shell, and payload of each microparticle being substantially identical to that of the other microparticles in the group. In other embodiments, a group of microparticles is provided, with the cap, the shell, and/or the payload of at least one microparticle being different from that of another microparticle in the group.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Embodiments will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIGS. 1A-1B are perspective and cross-sectional views, respectively, of initial, intermediate, and release stages of a microparticle, according to one or more embodiments of the disclosed subject matter.

FIGS. 1C-1D are perspective and cross-sectional views, respectively, of initial and release stages of a multi-core microparticle, according to one or more embodiments of the disclosed subject matter.

FIGS. 1E-1F are perspective and cross-sectional views, respectively, of initial and release stages of a microparticle with a cap having a geometric design, according to one or more embodiments of the disclosed subject matter.

FIG. 2A is a perspective view of a microparticle with a shell formed of multiple materials, according to one or more embodiments of the disclosed subject matter.

FIG. 2B is a cross-sectional view of another microparticle with a shell formed of multiple materials, according to one or more embodiments of the disclosed subject matter.

FIG. 2C is a perspective view of a microparticle with a cap formed of multiple materials, according to one or more embodiments of the disclosed subject matter.

FIG. 2D is a cross-sectional view of another microparticle with a cap formed of multiple materials, according to one or more embodiments of the disclosed subject matter.

FIG. 2E is a perspective view of a microparticle having multiple shell and cap components to form separate cores, according to one or more embodiments of the disclosed subject matter.

FIG. 2F is a cross-sectional view of a microparticle with a shell having an arbitrary shape, according to one or more embodiments of the disclosed subject matter.

FIG. 2G is a perspective view of a microparticle assembly employing a single cap for multiple shells, according to one or more embodiments of the disclosed subject matter.

FIG. 2H is a cross-sectional view of a microparticle with a functionalized shell, according to one or more embodiments of the disclosed subject matter.

FIG. 3 is a process flow diagram of an exemplary method for fabricating a microparticle, according to one or more embodiments of the disclosed subject matter.

FIGS. 4A-4D are simplified diagrams showing an exemplary microparticle fabrication system during shell patterning, evacuation, core loading, and cap patterning stages, respectively, according to one or more embodiments of the disclosed subject matter.

FIG. 4E illustrates various stages in an exemplary fabrication of a multi-core microparticle, according to one or more embodiments of the disclosed subject matter.

FIG. 4F illustrates various stages in another exemplary fabrication of a multi-core microparticle, according to one or more embodiments of the disclosed subject matter.

FIG. 5A is a process flow diagram of a generalized method for use of microparticles, according to one or more embodiments of the disclosed subject matter.

FIG. 5B illustrates a generalized group of fabricated microparticles for use in particular applications, according to one or more embodiments of the disclosed subject matter.

FIG. 6A illustrates various stages of different microparticles in a common group to achieve desired concentration profiles based on payloads released from the microparticles, according to one or more embodiments of the disclosed subject matter.

FIG. 6B illustrates various stages of an exemplary self-repairing structure incorporating microparticles, according to one or more embodiments of the disclosed subject matter.

FIG. 6C illustrates various stages of an exemplary structure that has microparticles within or disposed on the structure, according to one or more embodiments of the disclosed subject matter.

FIG. 6D illustrates various stages of an exemplary battery incorporating microparticles therein, according to one or more embodiments of the disclosed subject matter.

FIG. 6E illustrates an exemplary configuration for remote activation of microparticles within or disposed on a structure, according to one or more embodiments of the disclosed subject matter.

FIG. 6F depicts a generalized example of a computing environment in which the disclosed technologies may be implemented.

FIG. 7A is an image from a computer-aided manufacturing (CAM) simulation of a fabrication process for forming a batch of microparticle shells.

FIG. 7B is a scanning electron microscopy (SEM) image of fabricated microparticle shells corresponding to FIG. 7A.

FIG. 7C is an image from a CAM simulation of a fabrication process for forming caps for the batch of microparticle shells.

FIG. 7D is an SEM image of fabricated microparticles corresponding to FIG. 7C.

FIG. 8A is a graph of measured orifice diameter of fabricated microparticle shells versus designed orifice diameter for various laser writing magnifications.

FIG. 8B is a graph of measured outer diameter of fabricated microparticle shells versus designed orifice diameter for various laser writing magnifications.

FIG. 8C is a graph of relative fluorescence intensity (RFI) versus designed orifice diameter for liquid loaded into the microparticle cores using a vacuum-loading method.

FIG. 8D is a graph of RFI versus designed orifice diameter for liquid loaded into the microfluidic cores using a microfluidic infusion method.

FIG. 8E is a graph of RFI versus designed orifice diameter for microfluidic particles with liquid retained in the cores by respective caps.

FIGS. 9A-9C are perspective, top, and side views of finite-element analysis simulations of payload release when using a cap without a geometric design for the microparticle.

FIGS. 9D-9F are perspective, top, and side views of finite-element analysis simulations of payload release when using a cap with a concentric design for the microparticle.

FIGS. 9G-9H are SEM images of fabricated caps with exemplary geometric designs.

### DETAILED DESCRIPTION

### General Considerations

For purposes of this description, certain aspects, advantages, and novel features of the embodiments of this disclosure are described herein. The disclosed methods and systems should not be construed as being limiting in any way. Instead, the present disclosure is directed toward all novel and nonobvious features and aspects of the various disclosed embodiments, alone and in various combinations and sub-combinations with one another. The methods and systems are not limited to any specific aspect or feature or combination thereof, nor do the disclosed embodiments require that any one or more specific advantages be present, or problems be solved. The technologies from any embodiment or example can be combined with the technologies described in any one or more of the other embodiments or examples. In view of the many possible embodiments to which the principles of the disclosed technology may be applied, it should be recognized that the illustrated embodiments are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one skilled in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person skilled in the art to have a more definitive construction, the numerical parameters set forth are approximations that may depend on the desired properties sought and/or limits of detection under standard test conditions/methods, as known to those skilled in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including 10% of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Unless explained otherwise, all technical and scientific terms used herein have the same meaning as commonly understood to one skilled in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Features of the presently disclosed subject matter will be apparent from the following detailed description and the appended claims.

### Overview of Terms

The following explanations of specific terms and abbreviations are provided to facilitate the description of various aspects of the disclosed subject matter and to guide those skilled in the art in the practice of the disclosed subject matter.

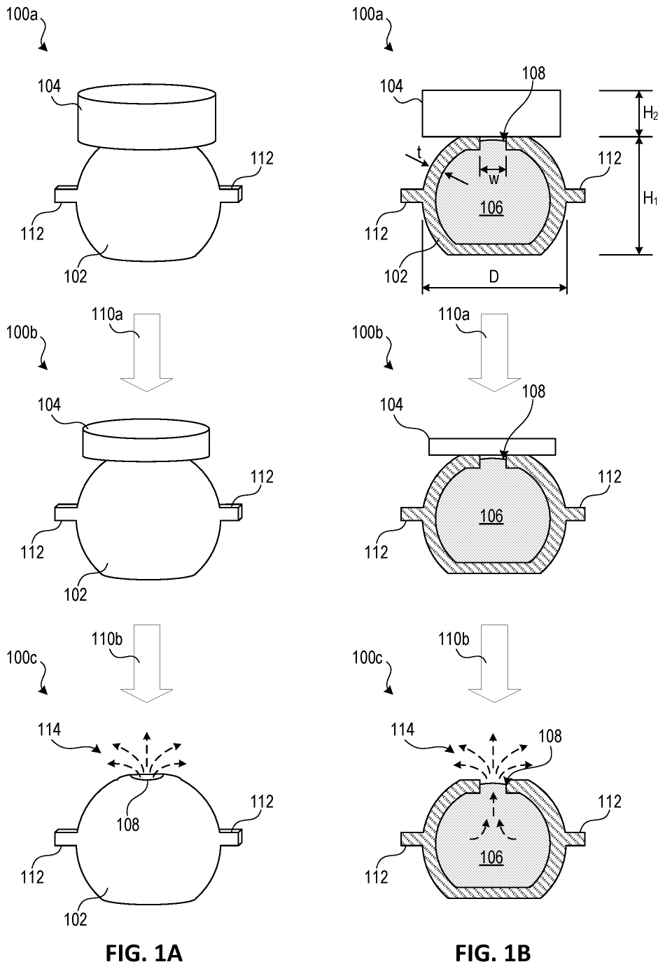

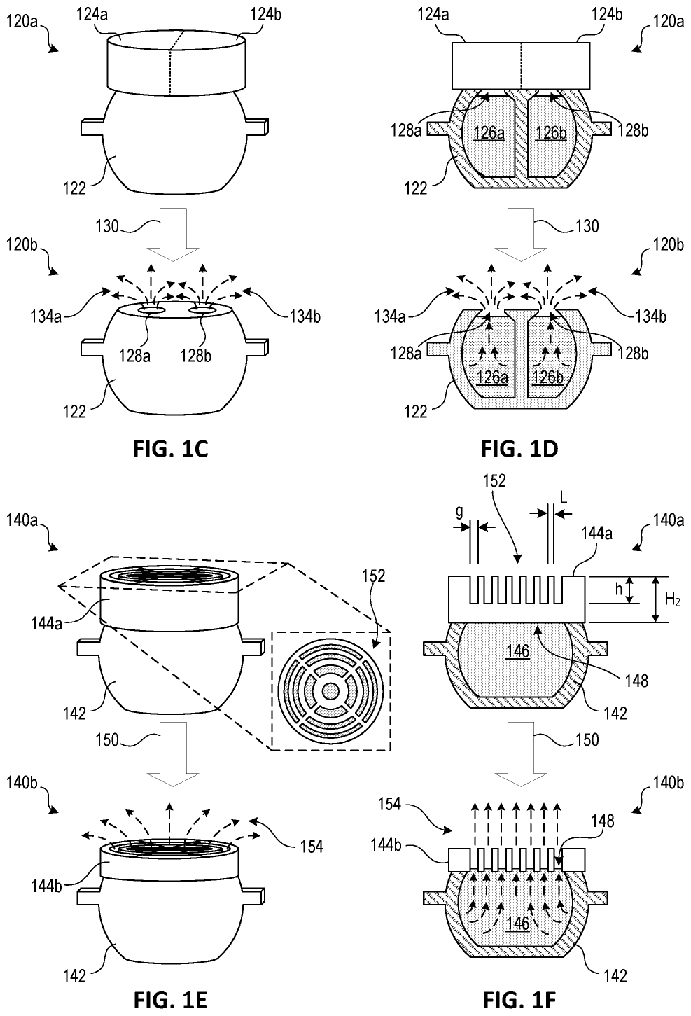

Heterogeneous microparticle (also referred to herein as particle, microcapsule, capsule, microcontainer, or container): An engineered particle having a maximum cross-sectional dimension (e.g., in FIG. 1B, the dimension D, the dimension resulting from H₁+H₂, or both) less than or equal to 300 μm (e.g., less than or equal to 200 μm, less than or equal to 100 μm, less than or equal to 50 μm, or less than or equal to 25 μm) and a payload within the particle that has a material composition and/or phase different than that of a shell or cap of the particle. In some embodiments, the payload retained within the particle is in a liquid phase. Although the term “microparticle” has been used herein for convenience, embodiments are not limited to dimensions in the microscale regime. Indeed, as capabilities of additive manufacturing techniques continue to evolve, engineered particles can be produced that have dimensions, precision, and/or feature sizes smaller than those explicitly stated above or elsewhere herein. Accordingly, in some embodiments, microparticles can have cross-sectional dimensions in the nanoscale regime (e.g., less than or equal to 1 μm, such as 100 nm or less, or even 10 nm or less), precision in the picoscale regime (e.g., less than or equal to 1 nm, such as 0.5 nm or less), and/or feature sizes in the picoscale regime (e.g., less than or equal to 1 nm, such as 0.5 nm or less).

Photomaterial: A photocurable material, or a material comprising a photoinitiator, that is capable of polymerizing (e.g., cross-linking) to form a solid structure upon exposure to a particular wavelength in the electromagnetic spectrum (e.g., X-ray, ultraviolet (UV), visible light, infrared (IR), etc.) or an electron (or other particle) beam, and/or exposure to a developer. In some embodiments, the material may undergo multi-photon polymerization (e.g., two-photon polymerization (TPP)) upon exposure to focused electromagnetic radiation (e.g., via direct laser writing (DLW)).

Triggering event: A continuous, periodic, or single-impulse condition in an environment to which the microparticle is exposed that causes the cap of a microparticle to dissolve, degrade, decay, disengage from the shell, or otherwise expose at least part of an orifice of the microparticle shell, so as to allow release of a payload therefrom. In some embodiments, the environmental condition can be a natural product of the environment (e.g., chemical composition of a biological environment, natural light within the environment, temperature of the environment, friction generated by normal use of a structure having the microparticle therein or thereon, etc.). Alternatively or additionally, the environmental condition can be an artificial stimulus provided to the environment (e.g., at the direction of a user or controller, applying energy or force to or within a structure having the microparticle therein or thereon, etc.). For example, the environmental condition can include but is not limited to stress, strain, pressure, fracture, friction, wear, electromagnetic radiation, electrical energy, magnetic energy, vibrational energy, ultrasonic energy, temperature, sensible heat, latent heat, pH, ionic concentration, chemical, biological environment, or any combination of the foregoing.

Biocompatible: A material that, when provided on or within a living host (e.g., human, animal, or otherwise), does not have toxic or injurious effects on the biological system of the host. Biocompatibility may be assessed in accordance with various regulatory guidelines, for example, “Use of International Standard ISO 10993-1,” U.S. Food & Drug Administration (FDA), FDA-2013-D-0350, September 2020, and “Biological evaluation of medical devices—Part 1: Evaluation and testing within a risk management process,” International Standard, ISO 10993-1, August 2018, both of which are incorporated herein by reference.

Biodegradable: A material capable of natural breakdown (e.g., on the order of hours, days, weeks, or months, depending on the application) into organic matter by a host in or on which the material is provided, or by microorganisms in an environment in which the material is provided. In some embodiments, the biodegradable material is a polymer, such as but not limited to bio-based plastics (e.g., polyhydroxyalkanoates (PHAs), polylactic acid (PLA), cellulose-based polymers, lignin-based polymers, starch blends, etc.), petroleum-based plastics (e.g., polyglycolic acid (PGA), polybutylene succinate (PBS), polycaprolactone (PCL), poly(vinyl alcohol) (PVA, PVOH), polybutylene adipate terephthalate (PBAT), etc.), or any combination thereof.

Geometric pattern or Geometric design: A pattern formed within a cap of a microparticle and configured to affect cap degradation characteristics (e.g., degradation rate, etc.), payload release characteristics (e.g., release rate, release direction, etc.), or both. In some embodiments, upon exposure to a triggering event, the geometric design of the cap forms a lattice or openwork (e.g., pattern of holes versus surrounding solid material), as opposed to a single opening for payload release. The pattern may be formed within a surface of the cap, such that the surface adopts a three-dimensional profile, as opposed to a planar surface of a simple cylindrical cap design or a single curved surface of a spherical cap design.

### Introduction

Using additive manufacturing techniques, heterogeneous microparticles can be fabricated, each microparticle having a payload sealed therein that can be controllably released in response to a triggering event. In some embodiments, at least part of the microparticle is formed by patterning of a photomaterial using focused electromagnetic radiation (e.g., one or more wavelengths in the X-ray, UV, visible light, or IR regimes) and/or a particle beam (e.g., electron, ion, neutron, or proton). For example, in some embodiments, a hollow shell of the microparticle can be formed by direct laser writing (DLW) to induce two-photon polymerization of the photomaterial in a point-by-point and/or layer-by-layer manner. In some embodiments, the payload can be introduced into a partially-formed microparticle (e.g., the hollow shell), for example, via diffusion, pressure filling, and/or vacuum-assisted filling. In some embodiments, the payload can then be enclosed within the microparticle by patterning of a same or different photomaterial to form a cap.

Referring to FIGS. 1A-1B, an example of an engineered heterogeneous microparticle at various stages of use is shown. In an initial state 100a (e.g., as-fabricated or as-deployed in a particular application), the microparticle comprises a hollow shell 102 and a cap 104 coupled to the shell 102. In some embodiments, the shell 102 (or parts thereof) can have a material composition that is different from that of the cap 104 (or parts thereof). Alternatively or additionally, the shell 102 (or parts thereof) and the cap 104 (or parts thereof) can have similar material compositions, but different configurations (e.g., different thicknesses, etc.) that result in different performance of the shell 102 and the cap 104 within the environment (e.g., a longer time for biodegradation for the shell than the cap).

In the illustrated example, the shell 102 has at least a partially spherical shape (e.g., with top and bottom ends partially flattened). However, other shapes are also possible according to one or more contemplated embodiments, such as but not limited to standard three-dimensional geometric shapes (e.g., cubic, polygonal, cylindrical, hemi-spherical, etc.), arbitrary shapes, or any combination thereof. In some embodiments, the shell 102 can have a diameter, D, (or a maximum cross-sectional dimension for non-spherical geometries) that is on the microscale, for example, less than 300 μm or less (e.g., 200 μm or less, 100 μm or less, 50 μm or less, or even 25 μm or less). The shell 102 can have a height, H₁, (e.g., in a direction perpendicular to D and/or in a direction along which orifice 108 extends) that is the same or different than the maximum cross-sectional dimension of the shell 102.

In the illustrated example, the shell 102 has protrusions 112 that can be used for alignment during the fabrication process. However, such alignment structures or markers are optional and can be omitted from some embodiments. In addition, although FIGS. 1A-1B illustrate two protrusions 112 and a particular shape for the alignment markers, additional or fewer markers and/or other shapes are also possible according to one or more contemplated embodiments. For example, additional protrusions can be provided in an orthogonal lateral direction (e.g., extending into and out of the page of FIGS. 1A-1B).

The shell 102 can comprise and/or define a core 106 and a flowpath or orifice 108, which extends through a wall of the shell 102. In the illustrated example, the sidewall of the shell 102 defining the core 106 can have a profile (e.g., shape in cross-section) that substantially matches that of the exterior of the shell 102, e.g., such that the thickness of the wall of the shell 102 is substantially constant (e.g., ˜2.5 μm). In some embodiments, the wall thickness, t, can be 10% or less than the maximum cross-sectional dimension of the shell 102. Alternatively, in some embodiments, the wall thickness can vary around the shell 102, such that the profile of the core 106 is different from that of the exterior of the shell 102. The orifice 108 and the core 106 can be in fluid communication, e.g., with the orifice extending directly from one end of the core (as shown), or with the orifice being indirectly connected to the core via an intervening flowpath (not shown).

In the illustrated example, the orifice 108 has a substantially cylindrical shape. However, other shapes are also possible according to one or more contemplated embodiments, such as but not limited to standard three-dimensional geometric shapes (e.g., cubic, polygonal, frustoconical, etc.), arbitrary shapes, or any combination thereof. In some embodiments, the orifice 108 can have a diameter (or maximum cross-sectional width for non-cylindrical geometries), w, that is slightly less than the maximum cross-sectional size of the shell (e.g., w=D−2t, for example, ˜20 μm), as low as a feature size limit of the additive manufacturing process (e.g., w of ˜100 nm), or anywhere in between. For example, in some embodiments, the orifice width can be given by 0.1α≤w≤0.6α (e.g., 2-12 μm inclusive, for a 25 μm diameter shell with 2.5 μm wall thickness), or by 0.2α≤w≤0.4α (e.g., 4-8 μm inclusive, for a 25 μm diameter shell with 2.5 μm wall thickness), where α=D−2t.

In some embodiments, the core 106 is filled (e.g., partially or fully) with a payload (e.g., liquid, solid, or both). The payload within the core 106 comprises one or more components that have a material composition different than that of the shell 102 and the cap 104. In some embodiments, at least one component of the payload can be provided to the core 106 (e.g., via orifice 108, prior to formation of cap 104) in a liquid phase and/or retained within the core 106 (e.g., after formation of cap 104 and/or in use) in a liquid phase. For example, in some embodiments, the payload within the core 106 can comprise a suspension or emulsion. In some embodiments, the payload can be provided to the core 106 in a liquid phase and then partially or fully converted to a solid (e.g., powder), for example, via freeze-drying. Alternatively or additionally, in some embodiments, the payload within the core 106 can include a living cellular organism (e.g., a fungus or bacteria) and/or a virus (e.g., a bacteriophage). In some embodiments, once loaded into the core 106, the living cellular organism or virus can be subjected to freeze drying, spray drying, etc., for example, to preserve the payload until subsequent release into the environment surrounding the microparticle.

Cap 104, which is formed over the orifice 108 and coupled (e.g., releasably or permanently) to the shell 102, can seal the orifice 108 from the surrounding environment, thereby retaining any contents within the core 106. In the illustrated example, the cap 104 has a solid cylindrical shape. However, other shapes are also possible according to one or more contemplated embodiments, such as but not limited to standard three-dimensional geometric shapes (e.g., cubic, polygonal, cylindrical, hemi-spherical, etc.), arbitrary shapes, or any combination thereof. In some embodiments, the cap 104 can have a diameter (or a maximum cross-sectional dimension for non-spherical geometries) that is the same as or different from the maximum cross-sectional dimension of the shell 102. In the illustrated example, the cap 104 can have a height, H₂, (e.g., in a direction perpendicular to D and/or in a direction along which orifice 108 extends) that is less than the height and the diameter of the shell 102, for example, a height, H₂, of ˜5 μm (e.g., when H₁ and/or D is ˜25 μm). Alternatively, in some embodiments, the height, H₂, of the cap 104 can be the same as or greater than the height and the diameter of the shell 102. In some embodiments, the maximum cross-sectional dimension of the microparticle can be the maximum cross-sectional dimension of the shell 102 (e.g., diameter D) or a combination of the heights of the cap 104 and the shell 102 (e.g., height H₁+height H₂).

The cap 104 can be constructed to maintain the seal of the orifice 108 until such time that release of the payload is desired, e.g., in response to a triggering event 110 (e.g., one or more natural or artificial environmental conditions, such as a chemical, force, or energy). For example, in response to a trigger, part or all of the cap 104 can be constructed to dissolve, degrade, decay, and/or disengage the cap 104 from the shell 102, thereby allowing the contents of the core 106 to be released into the surrounding environment via the orifice 108. For example, in FIGS. 1A-1B, exposure to triggering event 110a causes partial degradation (e.g., thinning) of the cap 104 while maintaining the structure of the shell 102 (e.g., without corresponding degradation of the shell 102, or with degradation that is otherwise insufficient to breach the shell wall to access the core 106), such that the microparticle progresses from the initial state 100a to an intermediate state 100b. In the intermediate state 100b, the orifice 108 can remain sealed by the partially-degraded cap, and the payload can be retained within the core 106. Further exposure to triggering event 110b can fully degrade the cap 104 while maintaining the structure of the shell 102, such that the microparticle progresses to the final state 100c. In the final state 100c, the orifice 108 is fully exposed from the cap, and the payload can be released into the surrounding environment (e.g., as shown by arrows 114) via the exposed orifice 108. Alternatively or additionally, in some embodiments, the cap 104 can be constructed to expose part of the orifice 108 while another part of the orifice 108 remains covered or obstructed by the cap (e.g., as discussed in further detail below with respect to FIG. 1F). Alternatively or additionally, in some embodiments, the microparticle can be constructed to proceed directly from the initial state 100a to the final state 100c (e.g., without intermediate state 100b where the cap 104 has a configuration different than either the initial state 100a or the final state 100c) in response to the triggering event, for example, where the triggering event causes the cap 104 to disengage from the shell 102.

In some embodiments, the payload can move from the core 106 to the surrounding environment via diffusion. Alternatively or additionally, the release of the payload from the core 106 can involve a pressure differential (e.g., squeezing force applied to shell 102 by surrounding environment), gravity (e.g., when the orifice 108 faces downward), centrifugal force (e.g., when the orifice 108 faces radially outward on a rotating structure), cohesion force (e.g., when a portion of the payload pulled through the orifice 108 by interaction with the environment pulls remaining portions with it), magnetic force (e.g., when the payload comprises a magnetic material), electro-osmotic forces (e.g., when an electric field is applied in the environment), any other transport-inducing mechanism, or any combination of the foregoing.

### Examples of Microparticle Configurations

In the example of FIGS. 1A-1B, the shell 102 of the microparticle comprises and/or defines a single core 106 with a corresponding single orifice 108 sealed by a single cap 104. However, in some embodiments, the microparticle can have multiple orifices and/or multiple caps (or portions of a single cap). For example, in some embodiments, the shell of the microparticle can be formed with orifices at opposite longitudinal ends of the single core, and a separate cap can seal each respective orifice (e.g., top and bottom caps). Alternatively or additionally, a single cap layer can be formed to cover multiple orifices, for example, as a second shell surrounding shell 102.

In some embodiments, the microparticle can have multiple cores, each with a respective orifice sealed by a corresponding cap (or portion of a single cap). For example, FIGS. 1C-1D show an exemplary multi-chamber microparticle. Similar to the above-described examples, the microparticle in its initial state 120a comprises a hollow shell 122 and a cap coupled to the shell 122, and the shell 122 (or parts thereof) can have a material composition and/or configuration (e.g., thickness) different than that of the cap. However, in the illustrated example, the shell 122 comprises and/or defines a first core 126a with first orifice 128a and a second core 126b with second orifice 128b. Although only two cores 126a, 126b are illustrated in FIGS. 1C-1D, additional cores (e.g., three or more in total) are also possible according to one or more contemplated embodiments. In some embodiments, each core (and its corresponding orifice) can have substantially the same size and shape as other cores (and their corresponding orifices) within the shell. For example, in FIGS. 1C-1D, cores 126a, 126b are reflection symmetrical. However, in some embodiments, one or some of the cores within the shell can have a shape and/or size different than another core within the shell, and/or one or some of the orifices can have a shape or size different than another orifice.

In some embodiments, each core is sealed by a respective cap (or a part of a cap). In the illustrated example, a first portion 124a of the cap is formed over the first orifice 128a and is coupled to the shell 122, and a second portion 124b of the cap is formed over the second orifice 128b and is coupled to the shell 122, thereby sealing the respective cores 126a, 126b from the surrounding environment. In some embodiments, the first cap portion 124a and the second cap portion 124b can be separately formed but otherwise constitute a common cap structure coupled to shell 122. Alternatively, in some embodiments, the first cap portion 124a and the second cap portion 124b can be commonly formed parts of the same cap structure (e.g., a unitary pattern in the additive manufacturing process). Alternatively, in some embodiments, the first cap portion 124a and the second cap portion 124b can be separate structures from each other (e.g., spaced from each other or with facing surfaces in contact).

In some embodiments, the payload contained in one core of the multi-chamber microparticle can be different from the payload contained in another core of the multi-chamber microparticle. For example, in some embodiments, the cores 126a, 126b can contain different drugs that when released together from the microparticle provide a synergistic or complementary effect. In another example, the cores 126a, 126b can separately store components that when combined (e.g., upon release and mixing in the surrounding environment) react to form a useful product (e.g., epoxy). Alternatively, in some embodiments, the payload contained in one core can be substantially the same as the payload contained in another core of the multi-chamber microparticle. For example, in some embodiments, the payload in cores 126a, 126b can be the same, but the release characteristics of the first cap portion 124a can be different than the second cap portion 124b, such that the payload from core 126a is released at a time different than that for the payload from core 126b.

In the illustrated example, each cap portion 124a, 124b can be constructed to maintain the seal of the corresponding orifice 128a, 128b until such time that release of the corresponding payload is desired, e.g., in response to triggering event 130. In some embodiments, the caps or cap portions sealing the orifices of the multi-chamber microparticle can have the same or similar configurations, e.g., substantially the same material composition and/or release characteristics, such that payloads contained in the respective cores can be released at substantially the same time in response to a triggering event. For example, in FIGS. 1C-1D, exposure to triggering event 130 causes degradation or detachment of each cap portion 124a, 124b while maintaining the structure of the shell 122 (e.g., without corresponding degradation of the shell 122, or with degradation that is otherwise insufficient to breach the shell wall to access the cores 126a, 126b), such that the multi-chamber microparticle progresses from the initial state 120a to final state 120b. In the final state 120b, each orifice 128a, 128b is fully exposed from its respective cap portion 124a, 124b, and the respective payloads can be released into the surrounding environment (e.g., as shown by arrows 134a, 134b) via the exposed orifices 128a, 128b. Alternatively, in some embodiments, the cap or cap portions can have different configurations, e.g., such that one cap portion degrades or detaches before the other cap portion, such that payloads contained in the respective cores can be released at different times in response to the same triggering event. Alternatively or additionally, in some embodiments, one cap or cap portion can be configured to respond to a triggering event different than that of another cap or cap portion.

In some embodiments, the cap (or portion thereof) can be constructed to regulate the release of the payload from the core of the microparticle, for example, by only partially exposing the orifice when subjected to the triggering event. For example, FIGS. 1E-1F show an exemplary microparticle with release-regulating cap. Similar to the above-described example, the microparticle in its initial state 140a comprises a hollow shell 142 and a cap 144a coupled to the shell 142, and the shell 142 (or parts thereof) can have a material composition and/or configuration (e.g., thickness) different than that of the cap 144a. The shell 142 can comprise and/or define a core 146 and a corresponding orifice 148 in fluid communication therewith.

The cap 144a can be patterned with a design (as contrasted with the solid cylinder of FIGS. 1A-1D), for example, recessed features formed within an exposed surface of the cap 144a. In some embodiments, such patterning of the cap 144a can be used when it is desirable for the cap to degrade over time in response to a triggering event (e.g., a biodegradable cap). In the illustrated example, the cap 144a is patterned with a geometric design 152, for example, an arrangement of suspended concentric rings in plan view. For example, the rings of the geometric design 152 can be formed by longitudinally-extending intervening portions (e.g., having a lateral dimension, L) separated from each other along a radial direction by recessed gaps (e.g., having a lateral dimension, g, and height, h). In some embodiments, the minimum dimensions for the lateral dimensions L, g in the geometric design 152 can be dictated by the feature size limitations of the corresponding additive manufacturing technique (e.g., 100 nm or greater).

In the initial state 140a, the cap 144a is formed over the orifice 148 and coupled (e.g., releasably or permanently) to the shell 142, thereby sealing the orifice 148 from the surrounding environment. The cap 144a can be constructed to maintain the seal of the orifice 148 until such time that release of the payload is desired, e.g., in response to a triggering event 150 (e.g., one or more natural or artificial environmental conditions, such as a chemical, force, or energy). For example, in FIGS. 1E-1F, exposure to triggering event 150 causes gradual degradation of the cap 144a while maintaining the structure of the shell 142 (e.g., without corresponding degradation of the shell 142, or with degradation that is otherwise insufficient to breach the shell wall to access the core 146), such that the microparticle progresses from the initial state 140a to regulated release state 140b. In the release state 140b, a thickness of the cap has been reduced by an amount greater than the depth, h, of the gaps in the geometric pattern 152 but less than the original full height, H₂, of the cap 144a. The gaps of the degraded cap 144b thus expose the underlying portions of orifice 148, while the concentric rings (which have been reduced in thickness by a corresponding amount) remain in place to block other portions of orifice 148.

In the release state 140b, the geometric pattern 152 can thus regulate the release of the payload through the exposed portions of the orifice 148 into the surrounding environment (e.g., as shown by arrow 154). In some embodiments, gap depth, h, and cap height, H₂, can be selected to dictate a time until initiation of payload release from the core and/or a time during which payload release is regulated by the longitudinally-extending portions (e.g., the concentric rings). In some embodiments, the cap pattern can provide regulation in terms of payload release timing (e.g., to avoid releasing the payload all at once), location of release (e.g., to avoid areas of concentration or to provide a desired concentration profile), or both.

Although a particular configuration for release regulation is illustrated in FIGS. 1E-1F, other configurations are also possible according to one or more embodiments. For example, in addition to or in place of the exposed surface of the cap being patterned, the undersurface of the cap (e.g., the surface facing the orifice) can include a pattern (e.g., with gaps instead in fluid communication with the orifice 148 instead of the surrounding environment). Alternatively or additionally, the orifice can be provided with its own pattern (e.g., a plurality of openings separated by intervening solid portions of the shell), although loading of a payload into the core through such a patterned orifice may be more difficult. Moreover, the suspended concentric ring design 152 in FIGS. 1E-1F is only one example of a geometric design, and other designs are also possible according to one or more contemplated embodiments. For example, the pattern applied to the cap can comprise a fractal geometry design, or the cap may be designed to form pores that have a distribution of geometric proportions that is fractal.

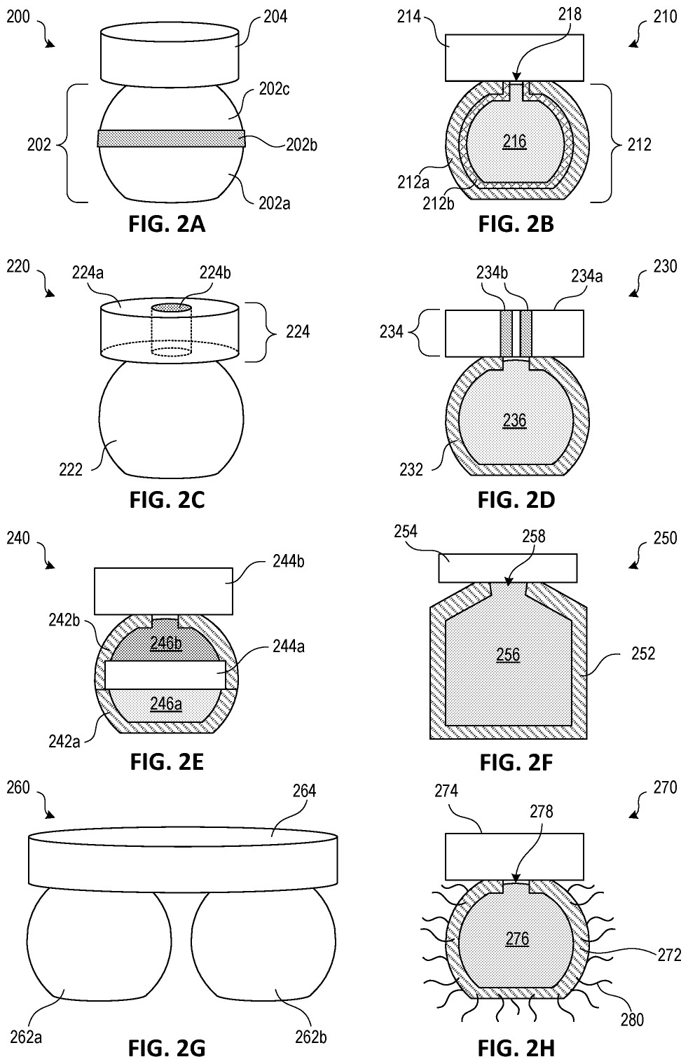

In the above-described examples, the shell of the microparticle can be formed of a single photomaterial and/or have a consistent material composition. However, in some embodiments, the shell of the microparticle can be formed of multiple photomaterials and/or have different material compositions. For example, FIG. 2A shows an exemplary microparticle 200 with a multi-material shell 202 and a cap 204. Similar to the other microparticles described above, the shell 202 can comprise and/or define a single continuous core (not shown) for containing a payload therein and an orifice (not shown) that is sealed by cap 204. However, the multi-material shell 202 can comprise or be formed by a plurality of sections 202a-202c, with at least one of the sections 202a-202c being formed of a different photomaterial and/or having a different material composition than another of the sections 202a-202c. Although three sections 202a-202c are shown in FIG. 2A, fewer or additional sections are also possible (e.g., only 2 or more than 3). Moreover, although FIG. 2A shows the shell sections 202a-202c with a particular orientation and shape, other orientations and/or shapes are also possible (e.g., as lateral stripes, as left-right halves, as inner and outer sections, etc.).

FIG. 2B shows another exemplary microparticle 210 with a multi-material shell 212 and a cap 214. Similar to the other microparticles described above, the cap 214 can be formed over orifice 218 and coupled to the shell 212 so as to seal a payload within the core 216. The multi-material shell 212 can comprise or be formed by a radially-inner section 212b that defines the core 216 (and optionally part of orifice 218) and a radially-outer section 212a that defines the exterior of the shell 212. The radially-inner section 212b can be formed of a different photomaterial and/or have a different material composition than that of the radially-outer section 212a. For example, the multi-material shell 212 of FIG. 2B may be useful in applications where the material of outer shell 212a is incompatible with the payload (e.g., inner shell 212b acts as protective barrier) or where material of inner shell 212b is designed to interact with the payload (e.g., where inner shell 212a comprises a nutrient to be consumed by a living cellular organism awaiting release from the core). Although two sections 212a-212b are shown in FIG. 2B, additional sections are also possible (e.g., 3 or more). Moreover, although FIG. 2B shows inner shell 212b extending over the entire interior of outer shell 212a, other configurations are also possible. In some embodiments, the inner shell can be disposed on or define a floor of the core (e.g., with sidewalls of the outer shell otherwise defining the bounds of the core), for example, to provide a functionalized floor where materials in the payload can be temporarily attached.

In the above-described examples, the cap of the microparticle can be formed of a single photomaterial and/or have a consistent material composition. However, in some embodiments, the cap of the microparticle can be formed of multiple photomaterials and/or have different material compositions. For example, FIG. 2C shows an exemplary microparticle 220 with a shell 222 and a multi-material cap 224. Similar to the other microparticles described above, the shell 222 can comprise and/or define a core (not shown) for containing a payload therein and an orifice (not shown) that is sealed by cap 224. However, the multi-material cap 224 can comprise or be formed by an inner cylindrical section 224b surrounded by an outer annular section 224a. The inner section 224b can be formed of a different photomaterial and/or have a different material composition than that of the outer section 224a. For example, the inner section 224b can be constructed to degrade faster than outer section 224a when exposed to the triggering event, to thereby expose part of the orifice while the outer section 224a remains in place. Although two sections 224a-224b are shown in FIG. 2C, additional sections are also possible (e.g., 3 or more). Moreover, although FIG. 2C shows the cap sections 224a, 224b with a particular orientation and shape, other orientations and/or shapes are also possible (e.g., as longitudinal sections, as lateral sections, as left-right halves, to provide the cap with a desired geometric design, etc.).

FIG. 2D shows another exemplary microparticle 230 with a shell 232 and a multi-material cap 234. Similar to the other microparticles described above, the shell 232 can comprise and/or define a core 236 for containing a payload therein and an orifice that is sealed by cap 234. The multi-material cap 234 can be formed from one or more second sections 234b disposed within and/or supported by a first section 234a. The first section 234a can be formed of a different photomaterial and/or have a different material composition than that of the second sections 234b. For example, the second sections 234b can be constructed to degrade when subjected to the triggering event, to thereby expose part of the underlying orifice. Meanwhile, in response to the same triggering event, the first section 234a can be constructed to degrade at a slower rate than the second sections 234b or to not degrade at all. For example, the multi-material cap 234 can be used to provide a geometric design for regulation of payload release (e.g., where second sections 234b correspond to the gaps between concentric rings in FIGS. 1E-1F).

In some embodiments, multiple components for the shell and/or the cap can be used to form custom microparticles of complex and arbitrary configurations. For example, FIG. 2E shows an exemplary microparticle 240 with a multi-component shell and multiple caps. In the illustrated example, a first shell component 242a comprises and/or at least partially defines a first core 246a, and a second shell component 242b comprises and/or at least partially defines a second core 246b and an orifice in fluid communication therewith. The microparticle 240 also includes an internal cap 244a that separates the first core 246a from the second core 246b, and an external cap 244b that is coupled to the second shell component 242b over the orifice to seal a payload within the second core 246b.

In some embodiments, the internal cap 244a can be configured to gradually degrade based on exposure to the payloads in core 246a, 246b. Alternatively or additionally, the internal cap 244a can be configured to degrade in response to external actuation (e.g., by application of force or energy). The degradation of the internal cap 244a can allow the contents of the cores 246a, 246b to mix together, for example, in preparation for deployment from the orifice of the second shell 242b once external cap 244b degrades or is otherwise removed in response to a triggering event. Alternatively, in some embodiments, the external cap 244b can be constructed to degrade after internal cap 244a, for example, to allow release of the payload in core 246a at a time after release of the payload in core 246b.

In many of the above-described examples, the components of the microparticle are either substantially spherical or cylindrical. However, embodiments of the disclosed subject matter are not limited to any particular shape for the microparticle overall or the individual components thereof. Rather, the components of the microparticle can adopt any arbitrary shape. For example, FIG. 2F shows an exemplary microparticle 250 having a shell 252 and a cap 254 sealing orifice 258. The shell 252 can comprise and/or define a core 256, which can have a shape in cross-sectional similar to or different from that of an exterior of the shell 252. In the illustrated example, the shell 252 has a non-spherical shape (e.g., six-sided polygonal); however, other shapes are also possible, including more complex geometries and arbitrary configurations (e.g., with longitudinal and/or lateral projections to engage the shell with the surrounding environment). Similarly, the cap 254 can have non-cylindrical shape, including more complex geometries and arbitrary configurations (e.g., with longitudinal and/or lateral projections to engage the cap with the surrounding environment).

In the above-described examples, a cap is coupled to and associated with a single shell to form a microparticle. However, in some embodiments, a single cap can be shared between shells, for example, to form a microparticle assembly. For example, FIG. 2G shows an exemplary microparticle assembly 260 where cap 264 is shared by separate shells 262a, 262b. Similar to the other microparticles described above, each shell 262a, 262b can comprise and/or define a respective core (not shown) for containing a respective payload therein and a respective orifice (not shown) that is sealed by cap 264. The size, shape, and/or material composition of shell 262a can be the same as or different than that of shell 262b.

In some embodiments, a payload contained within shell 262a is different than a payload contained within shell 262b. For example, the sharing of cap 264 by the different shells 262a, 262b can retain the shells 262a, 262b in proximity to each other for desirable combination of released payloads (e.g., components combining to form a foam or epoxy) once the cap degrades or is removed. Alternatively or additionally, in some embodiments, the sharing of cap 264 may allow for coordinated timing (e.g., substantially simultaneous) for the release of payloads from different shells 262a, 262b in response to a triggering event. Alternatively, in some embodiments, the payload contained within shell 262a can be substantially the same as the payload contained within shell 262b.

In some embodiments, one or more external surfaces of the microparticle can be functionalized to interact with the surrounding environment (or structure or entities therein). For example, FIG. 2H shows an exemplary microparticle 270 with a functionalized shell 272 and a cap 274. Similar to the other microparticles described above, the shell 272 can comprise and/or define a core 276 for containing a payload therein and an orifice 278 that is sealed by cap 274. However, the shell 272 can further comprise a plurality of ligands 280, for example, as part of its constituent photomaterial, coated thereon, or otherwise coupled thereto. Although the illustrated example of FIG. 2H has ligands 280 on exposed surfaces of the shell 272, in some embodiments, the ligands can be provided on only a portion of the shell 272 (e.g., only a side of the shell 272 opposite the orifice 278). In some embodiments, in addition to or in place of ligands 280 on the shell 272, the cap 274 can be functionalized, for example, with similar or different ligands as part of its constituent photomaterial, coated thereon, or otherwise coupled thereto. In some embodiments, the ligands 280 can be used to attach the shell 272 (and thereby the microparticle 270) to a receptor at a desired reaction site (e.g., a cell or tissue within a host for delivery of a drug payload thereto, for example, tumor-targeted delivery of a radiotherapy or other drug). Alternatively or additionally, the ligands 280 can be used to attach to a receptor on other microparticles.

Although the microparticles of FIGS. 1A-2H have been illustrated with particular cross-sectional shapes, embodiments of the disclosed subject matter are not limited thereto. Rather, any arbitrary 2-D shape or 3-D shape is possible for the structures, according to one or more contemplated embodiments. Moreover, although only a single microparticle is shown in FIGS. 1A-2F and 2H, in practical embodiments, a plurality of heterogeneous microparticles (e.g., a group, batch, dose, cohort, etc.) can be provided for use in a particular application. In some embodiments, each of the microparticles in the plurality can be substantially identical (e.g., having the same payload, the same release characteristics defined by the cap, the same core size, and/or cross-sectional dimensions). Alternatively, in some embodiments, at least some of the microparticles can be intentionally designed or tailored to have a payload, release characteristic, core size, and/or maximum cross-sectional dimension different than that of at least others of the microparticles.

### Examples of Methods and Systems for Fabrication of Microparticles

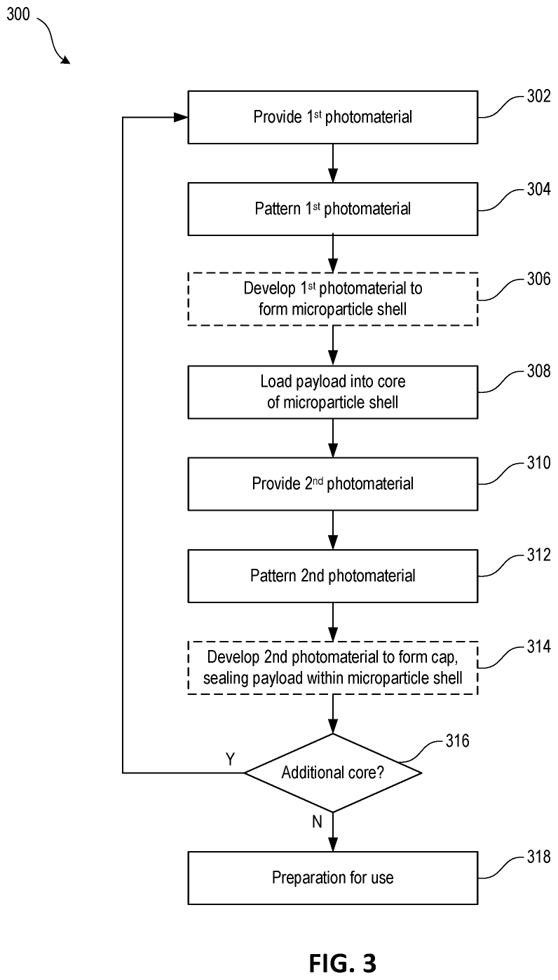

FIG. 3 illustrates an exemplary method 300 for fabricating one or more microparticles. The method 300 can be used to form a batch of microparticles (e.g., tens, hundreds, thousands, or millions of microparticles). In some embodiments, the size of the microparticles (e.g., shell diameter, D, or maximum cross-sectional dimension of the particle) can be substantially consistent across the batch, for example, such that a standard deviation of the size is 5% or less (e.g., 2% or less) of an average size of the microparticles in the batch (e.g., less than or equal to 0.5 μm for a particle size of ˜25 μm). Alternatively or additionally, in some embodiments, the method 300 can be used to form a batch of microparticles that are custom-designed with predetermined variations or differences (e.g., different shell size, different shell material, different core size, different cap size, different cap pattern, different cap material, different payload, or any combination thereof).

The method 300 can initiate at process block 302, where a first photomaterial is provided. In some embodiments, the provision of process block 302 can include forming the first photomaterial, e.g., by combining a desired material (e.g., biodegradable or biocompatible material) with a suitable photo-initiator. In some embodiments, the first photomaterial can be provided on a base or substrate, for example, by disposing atop the substrate or by flowing the first photomaterial into a channel (e.g., a microfluidic channel) formed on the substrate. In some embodiments, prior to or as part of process block 302, the substrate can be treated to improve adhesion of subsequently formed microparticle structures, for example, by silanizing the substrate. Alternatively or additionally, in some embodiments, prior to or as part of process block 302, a sacrificial layer can be formed on the substrate to assist in release of subsequently formed microparticle structures.

The method 300 can proceed to process block 304, where the first photomaterial is patterned to form microparticle shells. In some embodiments, the patterning can be performed by exposure of the first photomaterial in a point-by-point and/or layer-by-layer manner (e.g., writing) to one or more wavelengths in the electromagnetic spectrum (e.g., X-ray, UV, visible light, IR, etc.), for example, a focused beam from a substantially monochromatic electromagnetic radiation source (e.g., X-ray source, laser, etc.). The exposure can be effective to induce multi-photon polymerization (WIPP) within the first photomaterial. For example, in some embodiments, the patterning of the first photomaterial can be performed via oil-immersion direct laser writing (DLW), where an objective lens is used to focus femtosecond IR laser output onto the first photomaterial to induce two-photon polymerization (TPP) therein. Alternatively or additionally, the patterning of the first photomaterial can be via exposure to a particle beam (e.g., electron beam, neutron beam, proton beam, ion beam, etc.). Details regarding use of electron beams and/or X-rays beams for patterning of photomaterials can be found in “Electron and X-ray Focused Beam-Induced Cross-Linking in Liquids: Toward Rapid Continuous 3D Nanoprinting and Interfacing using Soft Materials,” ACS Nano, September 2020, 14(10); pp. 12982-92, which details are incorporated by reference herein.

After the patterning of process block 304, the method 300 can proceed to optional process block 306, where the exposed first photomaterial is developed to form shells of the microparticles. For example, the developing of process block 306 can include rinsing with one or more development chemicals, such as propylene glycol methyl ether acetate (PGMEA) and/or isopropyl alcohol (IPA). The developing of process block 306 can also include removing any unpatterned photomaterial (e.g., by rinsing or flushing), thereby leaving the fabricated shells on the substrate for subsequent processing. In some embodiments, depending on the first photomaterial, a separate develop may not be necessary, in which case process block 306 may simply comprise removing the unpatterned photomaterial in preparation for subsequent processing.

The method 300 can proceed to process block 308, where payloads are simultaneously loaded into the cores of the fabricated shells. In some embodiments, prior to or as part of the process block 308, the fabricated shells can be subjected to one or more rinsing steps prior to loading of the payloads, for example, to remove any unpatterned photomaterial and/or developer residue. In some embodiments, the payload can be provided in a liquid phase, for example, by disposing atop the substrate or by flowing the liquid payload into a channel (e.g., a microfluidic channel) formed on the substrate. In some embodiments, the payload can be loaded into the cores using a vacuum-assisted loading technique, where a pressure less than atmospheric pressure (e.g., a vacuum) is created within the cores and then allowing the liquid payload applied to an inlet of the channel (e.g., with an opposite outlet blocked) to passively fill any voids when the vacuum is released. Alternatively, the payloads can be loaded into the cores using any other technique, such as but not limited to infusion (e.g., applying positive pressure to drive the liquid payload in the channel into the cores) and/or diffusion.

After the loading of process block 308, the method 300 can proceed to process block 310, where a second photomaterial is provided. In some embodiments, prior to or as part of the process block 310, the payload-filled shells can be subjected to one or more rinsing steps prior to provision of the second photomaterial, for example, to remove any payload not contained within a corresponding core. In some embodiments, the provision of process block 310 can include forming the second photomaterial, e.g., by combining a desired material (e.g., biodegradable or biocompatible material) with a suitable photo-initiator. In general, the second photomaterial can have a material composition different from that of the first photomaterial. Alternatively, in some embodiments, the material composition of the first and second photomaterials can be substantially the same, for example, when the selected sizes and/or geometries of the microparticle components allow the cap to degrade or be removed while the shell is retained in response to the triggering event). In some embodiments, the second photomaterial can be provided by disposing over the formed microparticle shells on the substrate or by flowing the second photomaterial into a channel (e.g., a microfluidic channel) formed on the substrate.

The method 300 can proceed to process block 312, where the second photomaterial is patterned to form microparticle caps sealing the payloads within the respective shells. In some embodiments, prior to or as part of the process block 312, an alignment can be performed (e.g., based on alignment markers of the fabricated shells) to ensure that the patterned caps are properly formed upon the corresponding shells. In some embodiments, the patterning can be performed in a manner similar to process block 304, for example, by exposure of the second photomaterial in a point-by-point and/or layer-by-layer manner to one or more wavelengths in the electromagnetic spectrum (e.g., X-ray, UV, visible light, IR, etc.) and/or a particle beam. The exposure can be effective to induce MPP within the second photomaterial. For example, in some embodiments, the patterning of the second photomaterial can be performed via in situ DLW, where an objective lens is used to focus femtosecond IR laser output onto the second photomaterial contained within a microchannel on the substrate, so as to include TPP therein.

After the patterning of process block 312, the method 300 can proceed to optional process block 314, where the exposed second photomaterial is developed to form the caps of the microparticles. For example, the developing of process block 314 can include rinsing with one or more development chemicals, such as PGMEA and/or IPA. The developing of process block 314 can also include removing any unpatterned second photomaterial (e.g., by rinsing or flushing), thereby leaving the fabricated microparticles on the substrate for subsequent processing. In some embodiments, depending on the second photomaterial, a separate develop may not be necessary, in which case process block 314 may simply comprise removing the unpatterned photomaterial in preparation for subsequent processing.

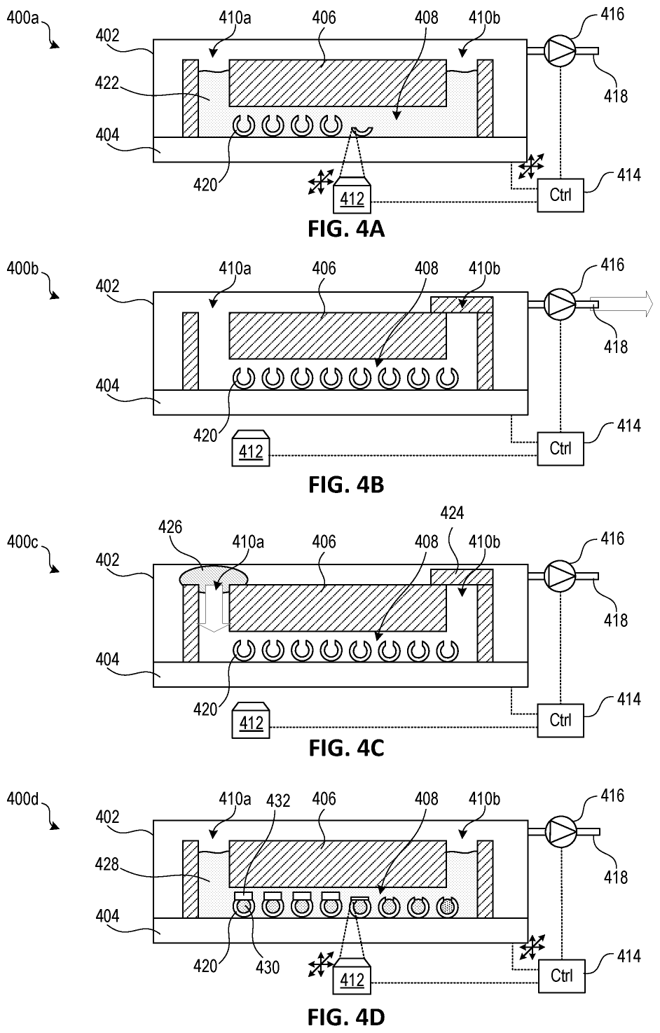

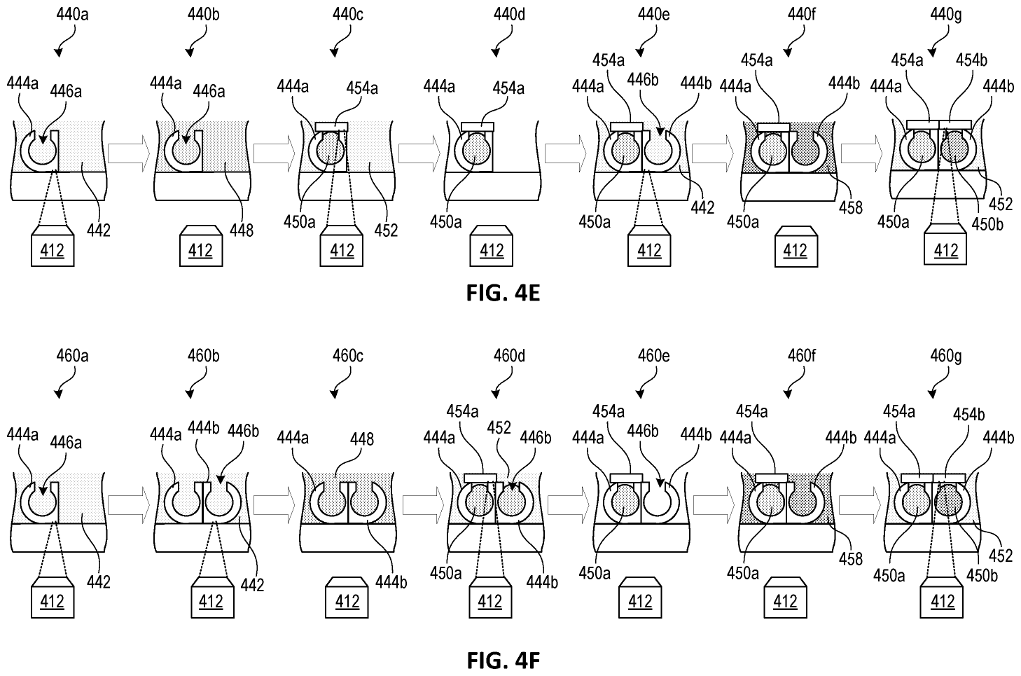

The method 300 can proceed to decision block 316, where it is determined if one or more additional cores are desired for each microparticle (e.g., to form a multi-chamber microparticle, such as in FIGS. 1C-1D). If additional cores are desired, some or all of process blocks 302-314 can be repeated to form additional cores of the microparticles and to seal respective payloads therein (for example, as described below with respect to FIG. 4E). In some embodiments, some or all of process blocks 302-314 can be repeated or performed out of order, for example, to form multiple shell portions defining multiple cores that are sequentially filled and capped (for example, as described below with respect to FIG. 4F).

If additional cores are not desired at decision block 316, the method 300 can proceed to process block 318, where the fabricated microparticles are prepared for subsequent use. In some embodiments, prior to or as part of process block 318, the microparticles can be released from the substrate, for example, by dissolving or etching a previously-formed sacrificial layer and/or the substrate itself. Alternatively or additionally, prior to or as part of process block 318, the microparticles can be subjected to freeze-drying, for example, to convert liquids in the cores into powders.

In some embodiments, the preparation of process block 318 can include combining the fabricated microparticles with microparticles fabricated in one or more other batches (e.g., in a different iteration of method 300). For example, the microparticles from one of the fabrication batches can have payloads and/or release characteristics different than that of microparticles in the other batch. In some embodiments, the preparation of process block 318 can include disposing the microparticles within a conveyance, for example, a solution for infusion (e.g., intravenous, etc.) or injection (e.g., intramuscular, subcutaneous, etc.) into a patient, a pill for ingestion by a patient, a paint or coating for disposing on a structure, a material precursor (e.g., polymer or polymer component) for forming as an integral part of a structure, etc. Alternatively or additionally, the preparation of process block 318 can include disposing microparticles within or on a patient (e.g., human, animal, or other living creature) or a structure (e.g., vehicle, building, casing, civil infrastructure, machinery, biological implant, furniture, any component thereof, etc.).

Although some of blocks 302-318 of method 300 have been described as being performed once, in some embodiments, multiple repetitions of a particular process block may be employed before proceeding to the next decision block or process block. In addition, although blocks 302-318 of method 300 have been separately illustrated and described, in some embodiments, process blocks may be combined and performed together (simultaneously or sequentially). Moreover, although FIG. 3 illustrates a particular order for blocks 302-318, embodiments of the disclosed subject matter are not limited thereto. Indeed, in certain embodiments, the blocks may occur in a different order than illustrated or simultaneously with other blocks. For example, patterning of process block 304 can form shells with multiple cores, which can be sequentially filled and capped by repeating process blocks 308-314 for each core.

In some embodiments, some or all of the method 300 of FIG. 3 can be performed by existing or customized additive manufacturing systems. For example, FIGS. 4A-4D illustrates operation of an exemplary setup for microparticle fabrication via DLW and vacuum-assisted loading. The setup can include a vacuum chamber 402 with an exhaust port 418, a vacuum pump 416 operatively coupled to the vacuum chamber exhaust port 418, illumination optics 412 (with corresponding laser source, not shown), positioning stages (e.g., translation or rotation stages) for moving the illumination optics 412 and/or the substrate 404 in one, two, or three dimensions with respect to each other, and a controller 414 operatively coupled to the illumination optics 412, a laser source (e.g., femtosecond laser), the positioning stages, and/or the vacuum pump 416 for controlling respective operations thereof.

For example, the illumination optics 412 can comprise an objective lens via which laser illumination is focused through the substrate 404 into the photomaterial within the vacuum chamber 402. Thus, in some embodiments, the substrate 404 (or a portion thereof) and/or the vacuum chamber 402 (or a portion thereof) can be substantially transparent to a wavelength of the laser illumination. A cover structure 406 (e.g., polydimethylsiloxane (PDMS) layer) can be releasably coupled (e.g., via thermal bonding) to the substrate 404 and can form a microchannel 408 (e.g., having a thickness in a direction perpendicular to the build plane and/or the substrate 404 less than or equal to 300 μm) with first and second ports 410a, 410b. The microchannel 408 can be sized and shaped to allow multiple microparticles to be formed in a single fabrication batch.

Referring to FIG. 4A, a shell patterning stage 400a is shown. A first photomaterial 422 is provided on or over the substrate 404, for example, by flowing into the microchannel 408 via one or both ports 410a, 410b. The controller 414 controls the positioning stages to move the focus of illumination optics 412 in a point-by-point and layer-by-layer manner within the first photomaterial 422 and controls operation of the laser to expose the first photomaterial at each focus (e.g., to cause TPP of the exposed photomaterial), thereby forming a shell 420 on substrate 404. As shown in FIG. 4A, multiple shells 420 can be formed during the shell patterning stage 400a, for example, by sequentially scanning the focus through the first photomaterial 422 on the substrate 404. After forming the shells 420, any undeveloped first photomaterial 422 can be removed from the substrate 404, for example, by flushing or rinsing the microchannel 408.

The setup can transition to the evacuation stage 400b of FIG. 4B. For example, port 410b of microchannel 408 can be closed by seal 424, and the controller 414 can control vacuum pump 416 to evacuate the vacuum chamber 402. As shown in the core loading stage 400c of FIG. 4C, a liquid phase material 426 can then be provided to uncovered port 410a, and the controller 414 can control exhaust port 418 to release the vacuum within the vacuum chamber 402. The resulting pressure differential passively drives the liquid material 426 into the microchannel 408 and then into the cores of the shells 420 via the exposed orifices thereof.

To seal the liquid material 426 within the cores, the setup can transition to the cap patterning stage 400d of FIG. 4D. A second photomaterial 428 is provided on or over the substrate 404, for example, by flowing into the microchannel 408 via one or both ports 410a, 410b. In a manner similar to the shell patterning, the controller 414 controls the positioning stages to move the focus of illumination optics 412 in a point-by-point and layer-by-layer manner within the second photomaterial 428 and controls operation of the laser to expose the second photomaterial at each focus (e.g., to cause TPP of the exposed photomaterial), thereby forming a cap 432 that seals the liquid payload 430 within the corresponding shell 420. After forming the complete microparticles, any undeveloped second photomaterial 428 can be removed, for example, by flushing or rinsing the microchannel 408.

In some embodiments, portions of the manufacturing system may be employed during only some of the fabrication stages. For example, the cover structure 406 can be provided only for evacuation stage 400b, core loading stage 400c, and cap patterning stage 400d, while the shell patterning stage 400a can be performed within a microchannel 408 being in place. Alternatively or additionally, the shell patterning stage 400a, the core loading stage 400c, and the cap patterning stage 400d can be performed outside of the vacuum chamber 402, with only the evacuation stage occurring in the vacuum chamber 402. In such configurations, the patterning writing components (e.g., illumination optics 412, controller 414, and positioning stages for the optics and/or substrate) can be considered as a separate system from the vacuum system (e.g., vacuum chamber 402, vacuum pump 416, and exhaust port 418), with the assembly of substrate 404 and cover structure 406 being movable between the separate systems.

Although the setup of FIGS. 4A-4D has been illustrated forming single-chamber microparticles, similar setups can be employed to form multi-chamber microparticles as well. For example, FIG. 4E illustrates exemplary stages in forming a multi-chamber microparticle. At stage 440a, a first photomaterial 442 can be introduced and patterned by focused radiation from optics 412 to form a first shell portion 444a with respective first core 446a. At subsequent stage 440b, undeveloped photomaterial 442 can be removed, and a first payload material 448 can be introduced into the first core 446a (e.g., via vacuum-assisted loading or another loading technique). At subsequent stage 440c, excess first payload material can be removed, and a second photomaterial 452 can be introduced and patterned by focused radiation from optics 412 to form a first cap portion 454a sealing payload 450a within the first core. At subsequent stage 440d, undeveloped photomaterial 452 can be removed in preparation for forming the next portions of the microparticle.

At subsequent stage 440e, the first material 442 can be re-introduced and patterned by focused radiation from optics 412 to form a second shell portion 444b with respective second core 446b. At subsequent stage 440f, undeveloped photomaterial 442 can be removed, and a second payload material 458 can be introduced into the second core 446b (e.g., via vacuum-assisted loading or another loading technique). In some embodiments, the second payload material 458 can be different than the first payload material 446, for example, having a different material composition and/or concentration. At subsequent stage 440g, excess second payload material can be removed, and the second photomaterial 452 can be re-introduced and patterned by focused radiation from optics 412 to form a second cap portion 454b sealing payload 450b within the second core. Any undeveloped photomaterial can be removed, and the stages repeated (e.g., stages 440e-440g) to form additional cores within the same microparticle. Alternatively, if no additional cores are desired, the microparticle can then be used or prepared for use in a particular application.

FIG. 4F illustrates exemplary stages of another approach for forming a multi-chamber microparticle. At stage 460a, a first photomaterial 442 can be introduced and patterned by focused radiation from optics 412 to form a first shell portion 444a with respective first core 446a. At subsequent stage 460b (which may be considered as part of, or a continuation of, stage 460a), the first photomaterial 442 can be further patterned by the focused radiation from optics 412 to form a second shell portion 444b with respective second core 446b. At subsequent stage 460c, undeveloped photomaterial 442 can be removed, and a first payload material 448 can be introduced into the first core 446a and the second core 446b (e.g., via vacuum-assisted loading or another loading technique). At subsequent stage 460d, excess first payload material can be removed, and a second photomaterial 452 can be introduced and patterned by focused radiation from optics 412 to form a first cap portion 454a sealing payload 450a within the first core.

However, after stage 460d, the second core 446b remains unsealed, such that the contents of the second core 446b (and any undeveloped photomaterial 452) can be removed (e.g., via rinsing or flushing, evacuation, or both) in subsequent stage 460e. At subsequent stage 460f, the second payload material 458 can be introduced into the second core 446b (e.g., via vacuum-assisted loading or another loading technique). At subsequent stage 460g, excess second payload material can be removed, and the second photomaterial 452 can be re-introduced and patterned by focused radiation from optics 412 to form a second cap portion 454b sealing payload 450b within the second core. Any undeveloped photomaterial can be removed, and the stages repeated (e.g., stages 460e-460g) to load additional cores (e.g., formed as part of stages 460a-460b) within the same microparticle. Alternatively, if no additional cores are desired, the microparticle can then be used or prepared for use in a particular application.

Although FIGS. 4E-4F illustrate and describe formation of a single multi-chamber microparticle, it should be apparent that the setups can be readily extended to formation of multiple microparticles in a single batch, for example, similar to the setup of FIGS. 4A-4D.

The fabrication method 300 of FIG. 3 and/or the batch fabrication setups of FIGS. 4A-4F can be adapted to provide a large number of heterogeneous microparticles with unique or custom characteristics, for example, by independent control of the patterning of each microparticle component in the batch (e.g., some with larger cores, some with larger caps, some with geometric design caps, etc.). For example, some embodiments may support pharmaceutical (or other industrial) development by unprecedented control of payloads and release options in a single print batch. For example, in some embodiments, a print run can produce as many as 5 million units in one hour, which in turn can yield 1000 or more doses. Each of these doses could represent a different release profile, since the cap thickness and geometry regulating the release may be digitally controlled and can be different for each microparticle. For example, in response to an emerging virus threat requiring accelerated testing of different doses, production of such test doses can be quickly and efficiently performed using the disclosed methods and/or systems.

### Examples of Methods and Systems for Usage of Microparticles

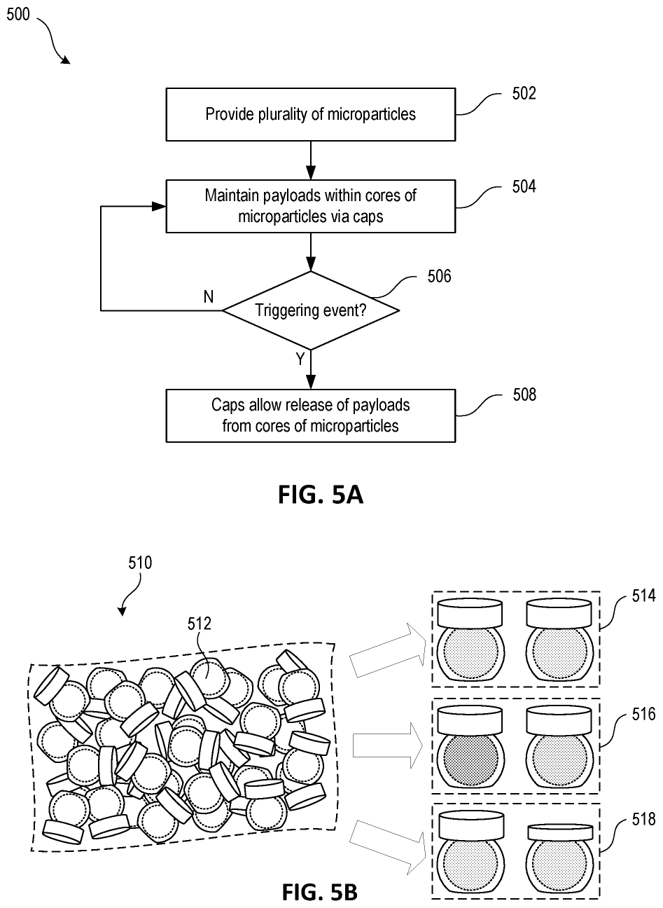

FIG. 5 illustrates an exemplary method 500 for use of microparticles. The method 500 can initiate at process block 502, where a plurality of microparticles can be provided. For example, in some embodiments, the provision of process block 502 can include all or part of fabrication method 300 of FIG. 3. In some embodiments, the microparticles in the provided plurality can be substantially identical to each other (e.g., same payload materials, same payload concentrations, same size, same release characteristics, etc.). Alternatively or additionally, at least some of the microparticles can be intentionally designed with different characteristics (e.g., having a different payload material, a different payload concentration, a different size, a different release characteristic, etc.) from other microparticles.

In some embodiments, the provision of process block 502 can include disposing the plurality of microparticles within or on a patient (e.g., human, animal, or other living creature), for example, via infusing, injecting, implantation, ingesting, etc. For example, since the microparticles are physicochemically stable, they can be delivered into the patient via any available pathway, including but not limited to nasal, ocular, buccal, or pulmonary delivery. Alternatively, in some embodiments, the provision of process block 502 can include disposing the plurality of microparticles within or on a structure (e.g., vehicle, building, casing, civil infrastructure, machinery, biological implant, furniture, any component thereof, etc.), for example, by injecting, impregnating, laminating, printing, coating, integrally forming with the structure, etc.

The method 500 can proceed to process block 504, where the caps of the microparticles continue to seal the payloads within the respective cores until such time that a sufficient triggering event is received at decision block 506. In response to sufficient triggering event exposure, the method 500 can proceed from decision block 506 to process block 508, where the payloads are released from the respective cores of the microparticles. Otherwise, the method 500 can return to process block 504, where the payload remains sealed within the respective microparticle by the cap.

In some embodiments, the triggering event can be a continuous, periodic, or single-impulse condition in an environment to which the microparticle is exposed that causes the cap of the microparticle to dissolve, degrade, decay, disengage from the shell, or otherwise expose at least part of an orifice so as to allow release of a payload therefrom. In some embodiments, the environmental condition can comprise a natural product of the environment (e.g., chemical composition of a biological environment, light within the environment, temperature of the environment, friction generated by normal use of a structure having the microparticle therein or thereon, etc.). Alternatively or additionally, in some embodiments, the environmental condition can comprise an artificial stimulus (e.g., at the direction of a user or controller) provided within or to the environment (e.g., a chemical, an energy (e.g., electromagnetic), or a force applied to or within a structure having the microparticle therein or thereon). For example, the environmental condition can include, but is not limited to, stress, strain, pressure, fracture, friction, wear, dissolution, electromagnetic radiation (e.g., X-ray, UV, visible, IR, radio frequency, etc.), electrical field, magnetic field, vibrational energy, ultrasonic energy, temperature, sensible heat, latent heat, pH, ionic concentration, chemical, biological environment (e.g., skin perspiration via salt, lactate, glucose, cortisol, etc.; saliva; blood; gastrointestinal fluids; etc.), or any combination of the foregoing.

In some embodiments, the triggering event can be used to initiate complete release of the contents of the microparticles (or one or more cores thereof) or to controllably regulate the rate of release of the contents of the microparticles. For example, the caps can regulate release using a geometric design to control rate of release and/or spatial distribution of release. Alternatively or additionally, the cap can be constructed to have a permeability and/or porosity that is controlled by the intensity of the triggering event (e.g., an applied electric field). In some embodiments, the payloads released during process block 508 can combine or react to form a product (e.g., an epoxy, a foam, etc.). For example, the payloads that combine or react can be released from the same microparticle (e.g., from different cores in a multi-chamber microparticle) and/or from different microparticles.

Referring to FIG. 5B, a generalized group 510 of fabricated microparticles 512 for use in particular applications is shown. In some embodiments, some or all of the microparticles 512 in the group 510 can be substantially identical. For example, as shown at first category 514, the microparticles can be substantially the same with respect to shell size (e.g., maximum cross-sectional dimension), core size (e.g., payload volume), shell material composition, release characteristics (e.g., cap size, cap material, cap pattern, etc.), and payload (e.g., material composition and/or concentration). Alternatively or additionally, in some embodiments, some or all of the microparticles 512 in the group 510 can have different payloads (e.g., material composition and/or concentration), for example as shown at second category 516. Alternatively or additionally, in some embodiments, some or all of the microparticles 512 in the group 510 can have different release characteristics (e.g., cap size, cap material, cap pattern, etc.), for example, as shown at third category 518. In some embodiments, microparticles according to any of the first through third categories 514-518 can be combined together in a common group for use in a particular application (e.g., with pairs of microparticles within the group falling into one or more of categories 514-518).

The ability to tailor the characteristics of the microparticles (e.g., payload and/or controllable release characteristics) can enable unique features and/or advantageous performance in a variety of applications. In some embodiments, the payloads can comprise a medicament (e.g., a liquid-phase active pharmaceutical ingredient (API)), nutrient, repellent, pesticide, protectant, adhesive, deterrent, fragrance, cosmetic, lubricant, component for forming any of the foregoing, or any combination of the foregoing. For example, in some embodiments, the payload may provide a therapeutic (e.g., drug or other treatment) that can be targeted to particular cells or tissues and/or controllably released in response to disease conditions and/or to provide desired concentrations. In some embodiments, the payloads can include a living cellular microorganism (e.g., fungus, bacteria) or a virus (e.g., bacteriophage), for example, encapsulated within the microparticle by using freeze drying, spray drying, in emulsions (e.g., ointments), etc.

For example, in some embodiments, the payloads can comprise a pesticide, such as N, N-Diethyl-meta-toluamide (DEET). The microparticles can be constructed for application to the skin of a user (e.g., human or other animal), clothing, netting, on structures in the field, etc. The microparticles can be constructed to release the payloads over time and/or in response to a particular triggering event (e.g., friction, warm body temperature, etc.). The retention of the pesticide within the microparticle until use is needed or desirable can help limit human exposure, avoid discomfort (e.g., greasy feeling, bad odors, skin irritation, etc.), and/or reduce environmental toxicity (e.g., minimize mammalian and/or fish toxicity).

In some embodiments, separate payloads from different microparticles in a group or from different cores in the same microparticle can be simultaneously released, for example, to provide synergistic or complementary effect. For example, the separate payloads can include different bacteriophages that are released to treat a bacterial infection in response to characteristics of the infection (e.g., heat, pH, etc.). By encapsulating the bacteriophages within the microparticles until ready for use, the concentration of the phages delivered to the site of the infection can be increased (thereby increasing effectivity), the shelf life can be improved, reproducible dosages can be provided, circulation time within the patient can be increased (e.g., for treating systemic infections, for prophylactic treatment, and/or treating intracellular infections), or any combination of the foregoing.

In another example, the separate payloads can simultaneously provide a desired drug cocktail, such as highly active antiretroviral therapy (HAART) for treating HIV infections (e.g., by providing a combination of emtricitabine and tenofovir disoproxil fumarate (F/TDF) in separate cores of a shell formed of poly(propylene fumarate) (PPF) and sealed by a cap formed of gelatin methacryloyl (GelMA)). In some embodiments, the caps of microparticles within the group or cap portions for the same multi-chamber microparticle can be constructed to respond to one or more different kinds of triggers. For example, in response to each type of trigger, a subset of the microparticles in the group can respond thereto (e.g., one subset that releases payloads in response to temperature exceeding a predetermined threshold, another subset that releases payloads in response to a pH outside of a predetermined range, etc.).

Alternatively or additionally, in some embodiments, payloads from different microparticles in a group or from different cores in the same microparticle can be released at different times, for example, to provide a predetermined release order or payload recipe (e.g., for an ordered release of ingredients and/or catalysts). In some embodiments, a single group of microparticles may contain different subsets, with each subset containing a particular payload. Each subset can have specific release characteristics (e.g., by changing cap material, cap thickness, and/or cap geometry), such that the release of each subset in the group occurs in a controlled order. For example, multi-drug microcarriers can provide multiple benefits as drug delivery vehicles, such as but not limited to the ability to program target release kinetics (e.g., constant, combined immediate-extended, or pulsatile multidrug release profiles), the ability to handle multiple, distinct liquid APIs, the ability to maintain stability at room temperature (e.g., ˜20° C.,) the ability to support on-demand carrier-specific (and thus, batch-specific) customization, or any combination of the foregoing.

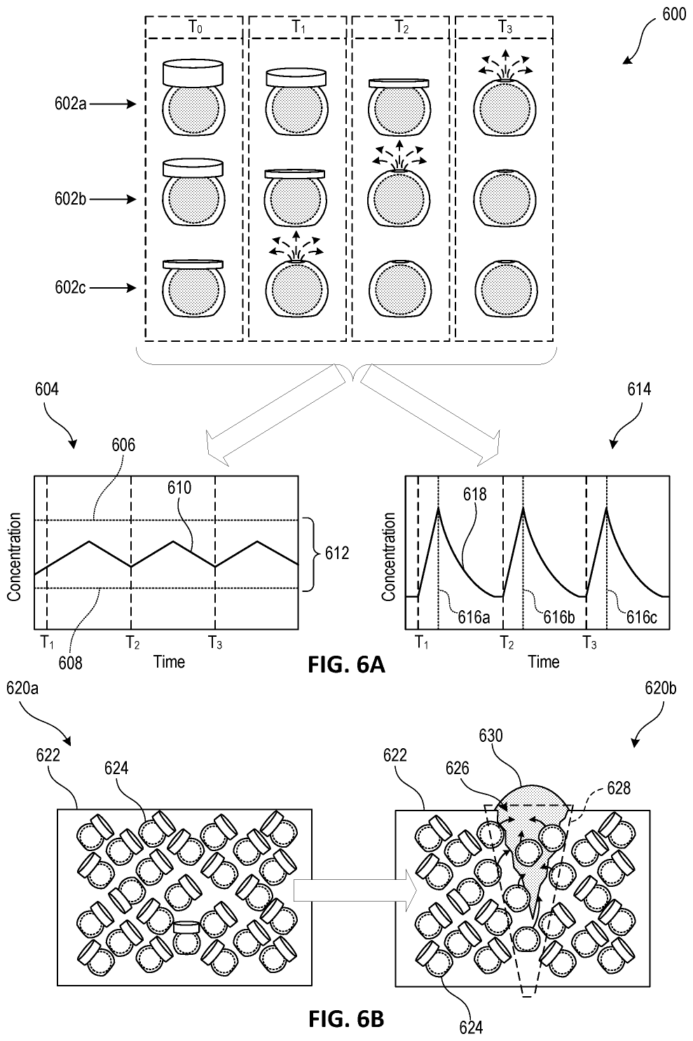

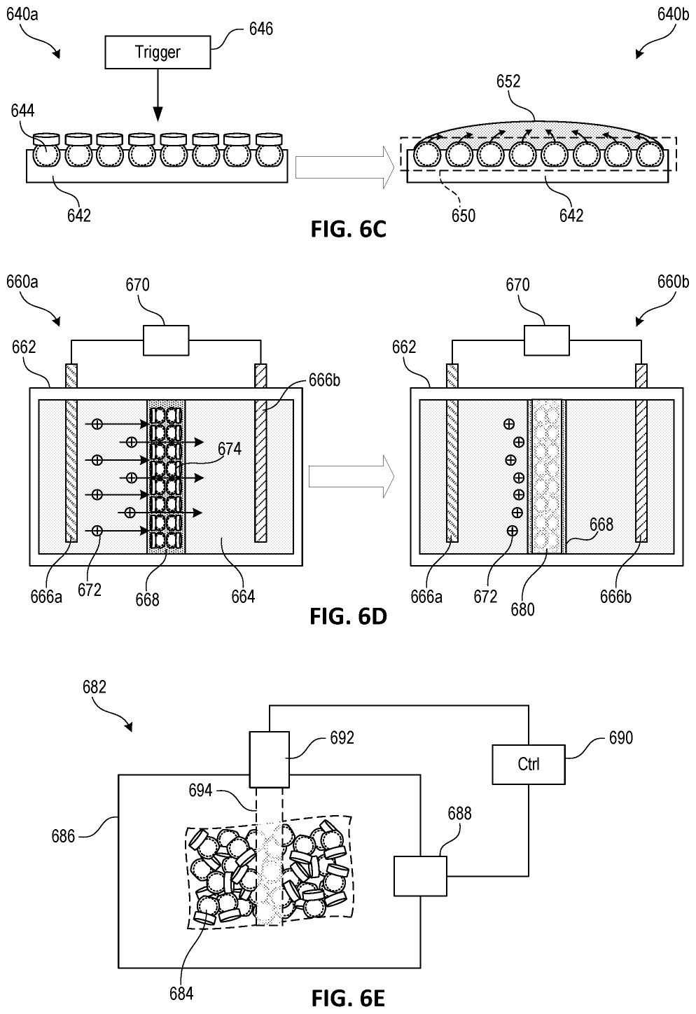

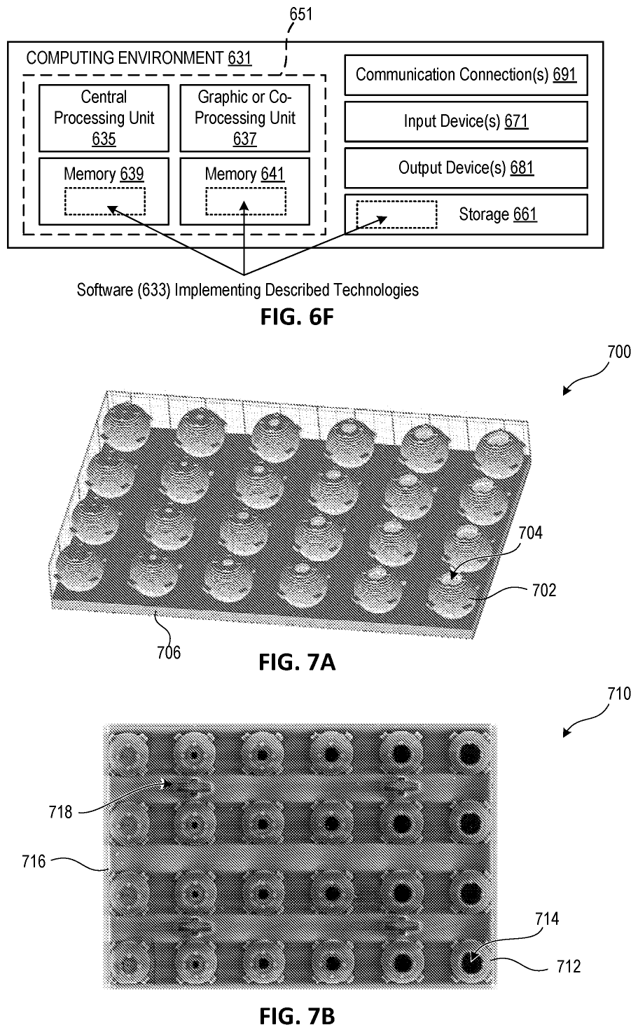

For example, FIG. 6A illustrates an exemplary use case where different microparticles are combined in a common group to achieve a desired release kinetics (e.g., constant, combined immediate-extended, or pulsatile release profiles) in a patient. For example, the group 600 of microparticles can include subsets 602a-602c with different release characteristics. Although three subsets 602a-602c are illustrated in FIG. 6A, fewer (e.g., 2) or greater (e.g., 4 or more) subsets can be provided within group 600. In the illustrated example, the different release characteristics are provided by virtue of biodegradable caps of different thicknesses. In particular, the caps of the first subset 602a are the thickest and thus take the longest time to release their payloads, while the caps of the third subset 602c are the thinnest and thus take the shortest time to release their payloads.

Alternatively, in some embodiments, the release characteristics can be set for each subset 602a-602c by a material composition of the cap (e.g., with the caps for subset 602a degrading more slowly than the caps for subset 602c), by a patterning of the cap (e.g., with the caps for subset 602a having a geometric pattern with a first gap depth, the caps for subset 602c having a geometric pattern with a second gap depth, and the second gap depth being greater than the first gap depth), by thickness of the caps, by trigger response (e.g., by having the caps of subset 602a and the caps of subset 602c respond to different environment conditions), or any combination of the foregoing.

In some embodiments, the payloads can be released at different times (e.g., T₁-T₃) to maintain a drug concentration within a desired range (e.g., in an effective range and/or away from a toxic range) or tailor drug concentrations within a patient to match expected activity of the patient's day. In one example, the group 600 of microparticles with different release characteristics can be used to provide a tailored concentration profile 610, for example, confined to a region 612 between an ineffective concentration 608 and a toxic concentration 606, as shown by graph 604. Such microparticles can be applied to drugs that historically have had issues balancing bioavailability and toxicity, for example, a small molecule tyrosine kinase inhibitor (smTKI), such as the cancer drug ibrutinib.

In another example, the group 600 of microparticles with different release characteristics can be used to provide a tailored concentration profile 618, for example, with concentration peaks corresponding to periods 616a-616c of greatest need, as shown by graph 614. Such microparticles can be applied, for example, to deliver a medication throughout the patient's day (or longer) via a single dose. For example, the drug could comprise Ritalin (methylphenidate hydrochloride), and the microparticles can be constructed to provide timed dosing precise enough to allow a patient to focus in the morning (e.g., time 616a), reduce the drug concentration around lunch (since Ritalin can interfere with appetite), increase the drug concentration for afternoon classes (e.g., time 616b), reduce the drug concentration around dinner, increase the drug concentration for homework (e.g., time 616c), and finally reduce the drug concentration for bedtime. Although the graph 614 in FIG. 6A shows the interval between peak concentration times 616a-616c (and corresponding release times T₁-T₃) being substantially equal, the intervals from one time to a successive time can instead vary (e.g., by appropriate design of cap release characteristics) to provide a customized or arbitrary release profile.

In some embodiments, separate payloads from different microparticles in a group or from different cores in the same microparticle can be simultaneously released, for example, to react or otherwise combine together to form a useful product, such as an epoxy, adhesive, paint, coating, lubricant, deterrent, stealth or specialty coating, seal, foam, etc. For example, the microparticles can encapsulate different substances that, when released from the cores and subsequently mixed, react to form an adhesive (e.g., a two-part epoxy). Such microparticles can be provided on or within a structure (e.g., printed on, injected into, coated on, and/or impregnated within the structure) to endow the structure with self-repairing and/or self-healing capabilities.

For example, FIG. 6B illustrates a structure (e.g., vehicle, building, casing, civil infrastructure, machinery, biological implant, furniture, any component thereof, etc.) that has a plurality of microparticles 624 disposed thereon or therein. At least some of the payloads of the plurality of microparticles 624 (whether separate microparticles or different cores within the same microparticle) can comprise separate components for forming an adhesive or epoxy. In the initial state 620a, the payloads can be retained within the respective microparticles. However, when subjected to a stress that causes formation of a structural defect 626 (e.g., a microcrack), a release state 620b can automatically evolve, where the payloads for at least some of the microparticles (e.g., in a region 628 proximal to the defect 626) are released to form an adhesive or epoxy 630 filling and/or repairing the defect 626 before it spreads. For example, in some embodiments, the stress and/or strain that produces the structural defect 626 can act as the trigger that causes caps of the microparticles to allow release of the respective payloads. Alternatively or additionally, an external triggering event (e.g., user or controller directed application of ultrasound or other mechanical energy) can be applied to the structure 622, for example, to effect repair of any defects 626 that may be present in the structure by the resulting epoxy 630 (e.g., as part of a periodic maintenance program).

In some embodiments, microparticles can be provided on a surface (e.g., as a coating, painting, etc.) or just below a surface (e.g., by forming a structure with the particles embedded therein), for example, to release payloads to the surface upon exposure to an appropriate triggering event. For example, FIG. 6C illustrates an exemplary configuration where microparticles 644 have been provided within or on a surface 642. In some embodiments, surface 642 can be an exterior surface of a user or patient (e.g., human or animal), clothing worn by the user or patient, or a structure (e.g., vehicle, building, casing, civil infrastructure, machinery, biological implant, furniture, any component thereof, etc.). In the initial state 640a, the payloads can be retained within the respective microparticles 644 by the corresponding caps. When region 650 of the surface 642 is subjected to an appropriate triggering event 646, release state 640b can automatically evolve, such that the caps of the microparticles 644 within the region 650 can allow the payloads within the microparticles to be released, for example, to form a layer, coating, application, or dosage 652 on or within surface 642. For example, the triggering event 646 can comprise stress, strain, pressure, fracture, friction, wear, electromagnetic radiation, temperature, sensible heat, latent heat, pH, ionic concentration, chemical, biological environment, or any combination thereof.

In some embodiments, the microparticles 644 can be substantially identical (e.g., same size, payload, and release characteristics). For example, the layer 652 formed by the payloads released from each microparticle 644 can comprise a medicament, nutrient, repellent, pesticide, protectant (e.g., sunscreen, surface protective film, etc.), an adhesive, a fragrance, a cosmetic, a lubricant, a living cellular organism, virus, combinations thereof, etc. Alternatively, in some embodiments, at least the payloads of some microparticles can be different than the remaining microparticles in or on surface 642, and the combination of the released payloads can result in a product (e.g., via mixing or reacting). For example, the layer 652 formed by the payloads released from each microparticle 644 can be a paint or coating for repairing or refreshing surface 642, and the triggering event for the payload release can be one or more conditions that cause or are indicative of damage to the surface 642 (e.g., friction, stress, strain, UV light, etc.). In another example, the layer 652 formed by the released payloads can be a lubricant (e.g., oil or grease), and the triggering event for the payload release can be one or more conditions that cause wear of the surface (e.g., friction) or are indicative of insufficient lubrication (e.g., vibration, heat, etc.).

In yet another example, the layer 652 formed by the released payloads can be a deterrent that provides an indication of intrusion or damage, and the triggering event for payload release can be one or more conditions related to the intrusion or damage (e.g., stress, strain, friction, UV, natural light, etc.). In still another example, the layer 652 formed by the released payloads can comprise a stealth or specialty coating, and the triggering event for payload release can be one or more native or biological conditions (e.g., in vivo) or artificial stimuli (e.g., application of energy or force). In some embodiments, a specialty coating can be a biologic coating, for example, a coating of drug-carrying microparticles on a stent (e.g., to provide the stent with drug-eluting capabilities) or a coating of drug-carrying microparticles on a surgical implant (e.g., to provide bone or wound repair). In another example, the layer 652 formed by the released payloads can comprise a skin cream or sunscreen, and the triggering event for payload release can be UV light, for example, to maintain effectivity while reducing user discomfort (e.g., minimizing a greasy feeling).

In yet another example, the layer 652 formed by the released payloads can comprise a failure arresting or sealing material, and the triggering event for payload release can be one or more conditions associated with such failure (e.g., heat, electrical current, etc.). In a further example, the layer 652 formed by the released payloads can comprise a foam (e.g., for fire control, to provide padding, etc.), and the triggering event for payload release can be one or more conditions related to a threat (e.g., heat from a fire, stress, strain, etc.). In some embodiments, the foam can be formed upon mixing of the release payloads, for example, via direct chemical reactions that greatly expand volume and/or by trapping air or other gases. In some embodiments, the foam can comprise a polymer (e.g., polyurethane). For example, the polymer foam can be formed by the combination of two different components (e.g., A and B) and a catalyst that are released from separate cores of the microparticles. Details regarding synthesis of such foams can be found in “The Role of Catalysis in the Synthesis of Polyurethane Foams Based on Renewable Raw Materials,” Catalysis Today, March 2014 223: pp. 148-56, which details are incorporated herein by reference. Such foams may expand dramatically on addition of the catalyst, the trigger of which can be tightly controlled by the microparticle release profile.

Referring to FIG. 6D, an exemplary application of microparticles 674 for failure mitigation of a battery 662 (e.g., a lithium-ion battery) is shown. Battery safety can be a considerable problem, with thermal runaway induced by short circuits resulting in temperatures of several hundred degrees Celsius and the likelihood of fire. Battery 662 can be provided with a polymeric separator 668 that acts as a permeable barrier between the electrodes 666a, 666b disposed within electrolyte 664. The polymeric separator 668 can have a plurality of microparticles 674 disposed thereon or embedded therein. In response to one or more triggering events indicative of a failure condition (e.g., heat, current, etc.), the battery 662 can progress from initial state 660a to release state 660b, where the microparticles 674 release their payloads. The released payloads can combine or react to form a sealing material 680 (e.g., epoxy) that renders the separator 668 substantially impermeable (e.g., with respect to ions 672), thereby reducing the progress of reactions within the battery that could lead to catastrophic failure. Alternatively or additionally, the released payloads can combine or react to form a fire-fighting foam 680, for example, to protect the battery against thermal runaway.

In some embodiments, the triggering event for the microparticles can be provided by an artificial stimulus, for example, exposure to a chemical, energy, or force at the direction of a user or a controller. For example, FIG. 6E illustrates an activation setup 682 where microparticles 684 have been provided within or on body 686. In some embodiments, body 686 comprises a user (e.g., human or animal) or a patient (e.g., human or animal being treated). Alternatively or additionally, in some embodiments, body 686 can comprise a structure (e.g., vehicle, building, casing, civil infrastructure, machinery, biological implant, furniture, any component thereof, etc.). Activation setup 682 can comprise an activation device 692 and a controller 690 operatively coupled thereto. For example, the activation device 692 can comprise one or more sources of electromagnetic radiation (e.g., UV, IR, RF, etc.), mechanical energy (e.g., acoustic or ultrasonic transducer), magnetic energy (e.g., electromagnet), electrical energy (e.g., voltage or current source), or any combination of the foregoing. The controller 690 can be configured to control the activation device 692 to direct force or energy 694 into the body 686 so as to cause the caps triggered by the force or energy 694 to allow release of payloads from the corresponding microparticles 684. In some embodiments, the activation setup 682 can optionally include a detector 688 for monitoring microparticles 684 and/or body 686. For example, the detector 688 can be a medical imaging modality configured to track when microparticles are proximal to a desired target within a patient (e.g., cancer cells). In some embodiments, the controller 690 can be configured to automatically activate microparticles 684 for payload release (e.g., via directed force or energy 694 from activation device 692) based on detection signals from detector 688. Alternatively or additionally, the controller 690 can be configured to accept user input regarding when and how to activate microparticle 684.

### Computer Implementation

FIG. 6F depicts a generalized example of a suitable computing environment 631 in which the described innovations may be implemented, such as aspects of method 300, controller 414, method 500, and/or controller 690. The computing environment 631 is not intended to suggest any limitation as to scope of use or functionality, as the innovations may be implemented in diverse general-purpose or special-purpose computing systems. For example, the computing environment 631 can be any of a variety of computing devices (e.g., desktop computer, laptop computer, server computer, tablet computer, etc.).

With reference to FIG. 6F, the computing environment 631 includes one or more processing units 635, 637 and memory 639, 641. In FIG. 6F, this basic configuration 651 is included within a dashed line. The processing units 635, 637 execute computer-executable instructions. A processing unit can be a general-purpose central processing unit (CPU), processor in an application-specific integrated circuit (ASIC) or any other type of processor. In a multi-processing system, multiple processing units execute computer-executable instructions to increase processing power. For example, FIG. 6F shows a central processing unit 635 as well as a graphics processing unit or co-processing unit 637. The tangible memory 639, 641 may be volatile memory (e.g., registers, cache, RAM), non-volatile memory (e.g., ROM, EEPROM, flash memory, etc.), or some combination of the two, accessible by the processing unit(s). The memory 639, 641 stores software 633 implementing one or more innovations described herein, in the form of computer-executable instructions suitable for execution by the processing unit(s).

A computing system may have additional features. For example, the computing environment 631 includes storage 661, one or more input devices 671, one or more output devices 681, and one or more communication connections 691. An interconnection mechanism (not shown) such as a bus, controller, or network interconnects the components of the computing environment 631. Typically, operating system software (not shown) provides an operating environment for other software executing in the computing environment 631, and coordinates activities of the components of the computing environment 631.

The tangible storage 661 may be removable or non-removable, and includes magnetic disks, magnetic tapes or cassettes, CD-ROMs, DVDs, or any other medium which can be used to store information in a non-transitory way, and which can be accessed within the computing environment 631. The storage 661 can store instructions for the software 633 implementing one or more innovations described herein.

The input device(s) 671 may be a touch input device such as a keyboard, mouse, pen, or trackball, a voice input device, a scanning device, or another device that provides input to the computing environment 631. The output device(s) 671 may be a display, printer, speaker, CD-writer, or another device that provides output from computing environment 631.

The communication connection(s) 691 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, radio-frequency (RF), or another carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java, Perl, any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

### Fabricated Examples and Experimental Results

To investigate the role of orifice diameter, w, in both the loading process and subsequent retention efficacy for the microfluidic core, computer-aided design (CAD) software was used to generate 3D models of six different shells corresponding to an outer diameter of 25 μm, a wall thickness of 2.5 μm, and orifice diameters, w, ranging from 2 μm up to 12 μm in increments of 2 μm. The models—designed at 10³×scale—were exported as an STL file and then imported into computer-aided manufacturing (CAM) software, which rescaled the model (i.e., 10³×smaller) for printing with an additive manufacturing printer (e.g., Nanoscribe Photonic Professional GT DLW 3D printer). Hatching and slicing printing parameters of 0.5 μm and 0.1 μm were used for the shells printed with the 25× and 63× objective lens, respectively. To examine fabrication discrepancies between the 25× and 63× objective lens, aliquots of their compatible photoresists—IP-S and IP-L 780, respectively—were dispensed onto distinct borosilicate glass substrates and loaded into the DLW printer in the oil-immersion configuration. To improve adhesion between the print and the substrate, the glass slide was silanized before printing by: (i) performing sequential rinses with acetone and isopropyl alcohol (IPA), (ii) O₂ plasma etching the slide at a pressure of 0.13 kPa with a power setting of 75 W for 45 s, and then (iii) submerging the slide in a 30 mL ethanol and 150 μL 3-(trimethoxysilyl)propyl methacrylate solution for 2 hr. Following the DLW process, the prints were developed using propylene glycol methyl ether acetate (PGMEA) and IPA, and then allowed to dry.

Brightfield and scanning electron microscopy (SEM) images and fluorescence micrographs were captured, and image quantifications were performed to measure the orifice and shell diameters from the SEM images as well as the particle-specific fluorescence intensities from the fluorescence micrographs. Experimental results are presented as mean±standard deviation (S.D.). The p values corresponding to differences in experimental measurements were calculated via unpaired two-tailed homoscedastic Student's t tests because the results associated with each design were not assumed to be distinct. Differences with p values less than 0.05 were considered statistically significant.

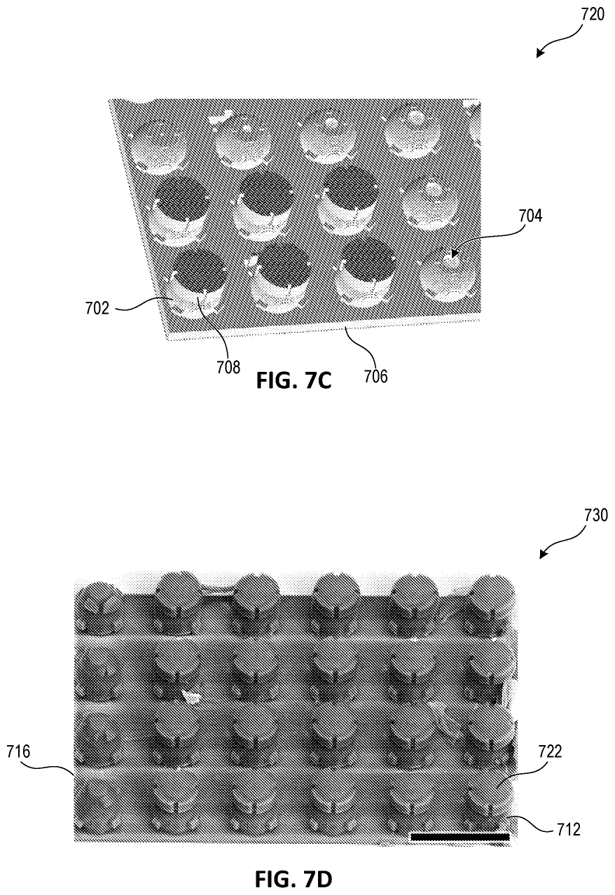

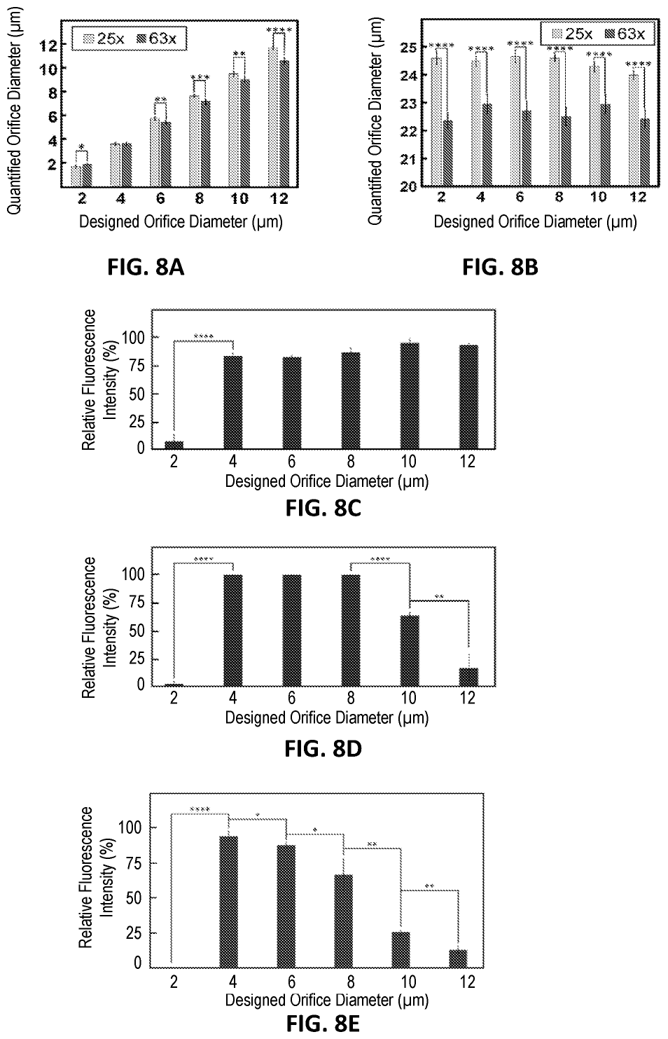

A CAM simulation 700 for DLW-based printing (25× objective lens) of outer shells 702 with different diameters for orifices 704 formed on a substrate 706 is shown in FIG. 7A. An SEM micrograph 710 of actual fabricated shell microstructures 712 with different diameters for orifices 714 formed on a substrate 716 (e.g., with alignment mark 718) is shown in FIG. 7B. Several differences between printing with a 25× and 63× objective lens were observed. The total print time increased from 20 s for the 25× objective lens up to 20 min for the 63× objective. Overall, the 63× objective appeared to produce slightly smaller orifice diameter values (i.e., 7.3±1.3% average difference) compared to the 25× objective for cases designed with an orifice width of 6 μm and larger (p<0.01), as shown in FIG. 8A (where *, **, ***, and *** denote p<0.05, p<0.01, p<0.001, and p<0.0001 statistically significant differences, respectively). From quantifying the outer diameters of the shells, however, it was found that DLW with the 63× objective lens consistently resulted in significantly more shrinkage compared to the 25× cases (p<0.0001 for each designed orifice diameter), as shown in FIG. 8B (where *, **, ***, and *** denote p<0.05, p<0.01, p<0.001, and p<0.0001 statistically significant differences, respectively). Despite the differences between the 25× and 63× objective lens results, the fabrication precision associated with a particular lens remained high (i.e., S.D.<0.4 μm and S.D.<0.25 μm for the 25× and 63× cases, respectively). Moreover, DLW fabrication results revealed high monodispersity for both microparticle shape and size (e.g., S.D.<0.4 μm and S.D.<0.25 μm for the 25× and 63× objective lens, respectively).

For subsequent loading of shells with respective payloads, a polydimethylsiloxane (PDMS) microchannel (50 μm in height; 250 μm in width) was formed and thermally bonded to the glass substrate, thereby enclosing the DLW-printed shells. Briefly, a negative master mold was printed on a Si substrate (25×25 mm²) using DLW in the Dip-in Laser Lithography (DiLL) configuration with the 25× objective lens. A 10:1 mixture of PDMS was poured onto the mold, degassed, thermally cured at 60° C. for 4 hr, removed from the master, and punched with inlet/outlet ports. The microchannel was aligned with respect to the printed shells, after which the PDMS was thermally bonded to the glass slide at 100° C. for 10 min. To passively load a target liquid of methylene blue-dyed DI water into the microchannel and DLW-printed shells, vacuum-assisted loading was employed. Tape was applied over the outlet (while the inlet remained unsealed) and the PDMS-on-glass device was placed in a vacuum chamber for 20 min. The device was then removed from the chamber, and a droplet of the target liquid was placed fully covering the inlet port. This process prompted the fluid to fill any voids within the PDMS device, including within the hollow capsules. Afterward, the tape was removed from the outlet port.

The microparticles were monitored using brightfield and fluorescence microscopy throughout the microfluidic vacuum-loading process and subsequent microfluidic infusion stages to provide insight into potential causes of error. The fluorescence results were quantified by calculating the relative fluorescence intensity (RN) corresponding to each microparticle as:

$$RFI = \frac{{x}_{i} - {x}_{min}}{{x}_{max} - {x}_{min}} \qquad ( 1 )$$

where xᵢ is the fluorescence intensity of a single microparticle, and xₘᵢₙ and xₘₐₓ are the minimum and maximum microparticle-specific fluorescence intensities for all of the microparticles, respectively. After the microfluidic vacuum-loading step, it was observed that the methylene blue-dyed DI water appeared to load effectively into the microparticles designed with orifice diameters of 4 μm, while microparticles with orifice diameters of 2 μm inhibited such loading, resulting in significantly reduced RFI values (p<0.0001), as shown in FIG. 8C (where *, **, ***, and **** denote p<0.05, p<0.01, p<0.001, and p<0.0001, statistically significant differences, respectively). Following the photomaterial loading process, however, only the microparticles designed with orifice widths ranging from 4 μm to 8 μm appeared to maintain the microfluidic core as the orifice widths greater than or equal to 10 μm exhibited evidence of core loss with associated significant reductions in RFI magnitudes (p<0.0001), as shown in FIG. 8D (where *, **, ***, and **** denote p<0.05, p<0.01, p<0.001, and p<0.0001, statistically significant differences, respectively).

To evacuate the fluid associated with the core loading step from the microchannel, rhodamine B-dyed glycerol was infused into the channel using a syringe, followed by a liquid-phase photomaterial (IP-L 780) for the cap. Using an in situ DLW (isDLW) approach, identical caps (25 μm in diameter; 5 μm in height) were printed with the 63× objective lens via the same protocols as those for the outer shells, with the caveat that an alignment step was needed to ensure full fluidic sealing of the printed caps atop the previously printed shells. To support this optical (manual) alignment step, the outer shells were designed with physical markers. After DLW-based printing of the caps, the print was developed with PGMEA and IPA.

A CAM simulation 720 of the fabrication results for isDLW-based printing of microparticle caps 708 is shown in FIG. 7C. The print time for one set of six caps using the 63× objective lens was approximately 90 s. An SEM micrograph 730 of fabrication results revealed significant printing errors associated with microparticles designed with orifice diameters of 2 μm. One potential basis for this error is that by preventing the core loading, any remaining air pockets within the core can lead to thermal effects that disrupt the DLW process and lead to print failures. Nonetheless, for all of the other microparticles with larger orifice diameters that yielded successful vacuum-loading of a microfluidic core, print failures of caps 722 were not observed, as reflected in FIG. 7D.

To investigate the role of the cap in core retention performance, rhodamine B-dyed IPA was infused into a microchannel comprising adjacent sets of microparticles with and without the DLW-printed caps. For the negative control case (i.e., without caps), detectable methylene blue-dyed fluid was not observed in the microparticles as the core locations instead appeared to be filled with the rhodamine B-dyed IPA. In contrast, the microparticles with the printed caps successfully sealed their corresponding microfluidic cores, thereby preventing undesired core-filling of the IPA. Both the optically observable and quantified results were similar to those following the photomaterial infusion step, but with slightly more pronounced reductions in fluorescence intensity for microparticles designed with orifice width greater than 4 μm, as shown in FIG. 8E (where *, **, ***, and **** denote p<0.05, p<0.01, p<0.001, and p<0.0001, statistically significant differences, respectively). Specifically, microparticles designed with orifice width equal to 4 μm exhibited the highest core retention performance with a mean RFI of 94.7±3.2%, as shown in FIG. 8E. Each 2 μm increase in orifice width led to statistically significant reductions up to an orifice width of 8 μm case (p<0.05), with an RFI of 66.7±12.2%, as shown in FIG. 8E. The cases with larger designed orifice width values, however, exhibited greater decreases in core retention performance (p<0.01), with mean RFIs of 25.4±1.8% and 13.0±2.5% for microparticles designed with orifice widths of 10 μm and 2 μm, respectively, as shown in FIG. 8E. These results suggest that the orifice widths of 4 μm may be best suited for liquid-core-shell microparticle manufacturing via the presented DLW-based methodology. Although the quantified experimental results for the microparticles and target fluids in this work revealed that orifice widths of 4 μm can yield the highest core retention performance of those investigated, changes in the outer shell diameter, orifice architecture, or target fluid will likely require additional experimentation to determine which geometries are best suited for a particular case.

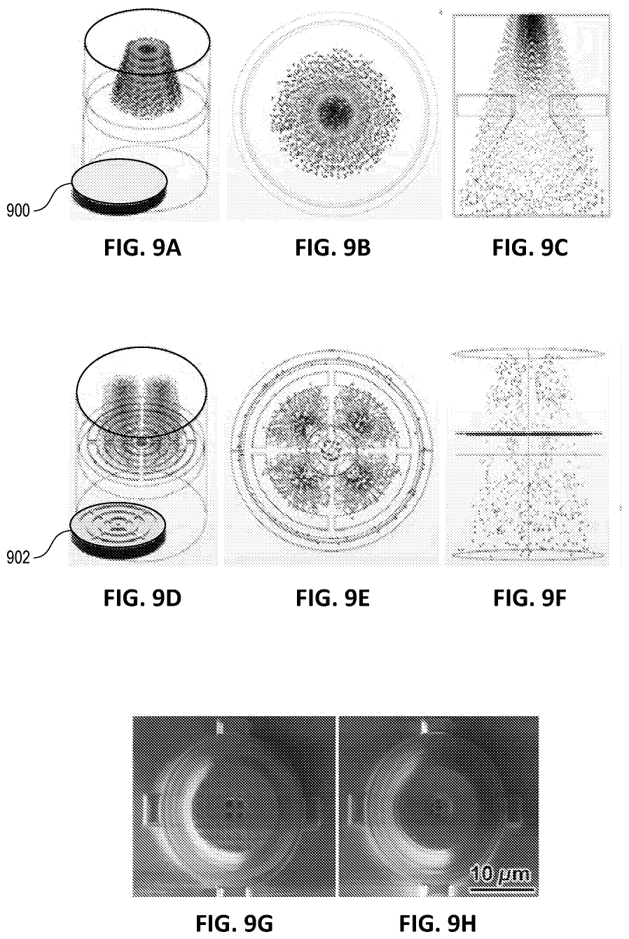

FIG. 9D illustrates an exemplary cap 902 for providing design-mediated core release, and FIG. 9A illustrates a uniform thickness cap 900. The cap 902 and cap 900 can both be biodegradable and printed via DLW; however, the key difference is that while the majority of the cap 902 is printed as a uniformly solid entity, the final top layer can be printed with a custom geometric design (e.g., concentric ring design). During the biodegradation, the cap 902 degrades leading up to the penultimate stage in which only the geometric design remains. As a result, the core is able to diffuse out of the shell, but with the diffusion behavior now physically modified by the residual geometry.

The transmission probabilities of 10 μm size particles through the uniform-thickness cap 900 and the geometric design cap 902 were modeled using finite-element analysis (FEA) simulations, as shown in FIGS. 9A-9C and 9D-9F, respectively. The FEA simulation results revealed that cap 902 with geometric design was able to significantly limit core diffusion properties compared to control cap 900 (no design). Preliminary DLW-based printing tests revealed high control of cap design features and pore sizes, as shown by the exemplary geometric designs of FIGS. 9G-9H.

### Additional Examples of the Disclosed Technology

In view of the above described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

Clause 1. A method comprising:
(a) providing a plurality of first heterogeneous microparticles, each first heterogeneous microparticle having a maximum cross-sectional dimension of less than or equal to 300 μm, each first heterogeneous microparticle comprising:
   a first shell defining a first core and a first orifice in fluid communication with the first core;
   a first payload disposed within the first core; and
   a first cap coupled to the first shell so as to seal the first orifice, the first cap and the first shell each comprising a respective cured photomaterial; and
(b) subjecting the plurality of first heterogeneous microparticles to a first triggering event, such that at least a portion of each first orifice is exposed from the respective first cap so as to release, through the exposed first orifice portion, the corresponding first payload from the first core of the first shell.
   Clause 2. The method of any clause or example herein, in particular, Clause 1, wherein, for at least one of the first heterogeneous microparticles, a material composition of the first shell is different than a material composition of the corresponding first cap.
   Clause 3. The method of any clause or example herein, in particular, any one of Clauses 1-2, wherein, for at least one of the first heterogeneous microparticles, the first shell is formed of a biocompatible material and the corresponding first cap is formed of a biodegradable or biocompatible material.
   Clause 4. The method of any clause or example herein, in particular, any one of Clauses 1-3, wherein, for at least one of the first heterogeneous microparticles, both the first shell and the first cap are formed of biodegradable materials.
   Clause 5. The method of any clause or example herein, in particular, any one of Clauses 1-4, wherein, in response to the first triggering event, the first cap degrades faster than the first shell.
   Clause 6. The method of any clause or example herein, in particular, any one of Clauses 1-5 wherein one or more of the first payloads comprises at least one liquid phase component within the respective first core.
   Clause 7. The method of any clause or example herein, in particular, any one of Clauses 1-6, wherein one or more of the first payloads comprises a medicament, a nutrient, a repellent, a pesticide, a protectant, an adhesive, a deterrent, a fragrance, a cosmetic, a lubricant, a component for forming any of the foregoing, or any combination of the foregoing.
   Clause 8. The method of any clause or example herein, in particular, any one of Clauses 1-7, wherein one or more of the first payloads comprises a living cellular organism or virus.
   Clause 9. The method of any clause or example herein, in particular, any one of Clauses 1-8, wherein one or more of the first payloads comprises a bacteriophage, a fungus, or a bacteria.
   Clause 10. The method of any clause or example herein, in particular, any one of Clauses 1-9, wherein one or more of the first payloads comprises a powder.
   Clause 11. The method of any clause or example herein, in particular, Clause 10, wherein the providing of (a) comprises forming the powder in situ within the corresponding first core by freeze drying.
   Clause 12. The method of any clause or example herein, in particular, any one of Clauses 1-11, wherein the first triggering event comprises exposure to one or more environmental conditions.
   Clause 13. The method of any clause or example herein, in particular, Clause 12, wherein the one or more environmental conditions comprise stress, strain, pressure, fracture, friction, wear, electromagnetic radiation, temperature, sensible heat, latent heat, pH, ionic concentration, chemical, biological environment, or any combination of the foregoing.
   Clause 14. The method of any clause or example herein, in particular, any one of Clauses 1-13, wherein the first triggering event comprises active application of energy or force to the plurality of first heterogeneous microparticles.
   Clause 15. The method of any clause or example herein, in particular, Clause 14, wherein the energy or force comprises electromagnetic radiation, electrical energy, magnetic energy, vibrational energy, ultrasonic energy, sensible heat, latent heat, or any combination of the foregoing.
   Clause 16. The method of any clause or example herein, in particular, any one of Clauses 1-15, wherein, for one or more of the first heterogeneous microparticles, the first cap is patterned such that, in response to the first triggering event, an area of the first orifice exposed from the first cap changes over time so as to regulate release of the first payload from the first core.
   Clause 17. The method of any clause or example herein, in particular, Clause 16, wherein one or more of the first payloads includes a drug.
   Clause 18. The method of any clause or example herein, in particular, any one of Clauses 16-17, wherein, for one or more of the first heterogeneous microparticles, a feature size of the pattern in the first cap is less than or equal to 100 nm.
   Clause 19. The method of any clause or example herein, in particular, any one of Clauses 16-18, wherein, for one or more of the first heterogeneous microparticles, the first cap has a geometric pattern.
   Clause 20. The method of any clause or example herein, in particular, any one of Clauses 16-19, wherein, for one or more of the first heterogeneous microparticles, the first cap has a fractal pattern.
   Clause 21. The method of any clause or example herein, in particular, any one of Clauses 1-20, wherein, for the plurality of first heterogeneous microparticles, a standard deviation of the maximum cross-sectional dimensions is less than or equal to 5% of an average of the maximum cross-sectional dimensions, or less than or equal to 2% of an average of the maximum cross-sectional dimensions.
   Clause 22. The method of any clause or example herein, in particular, Clause 21, wherein the standard deviation of the maximum cross-sectional dimensions within the plurality of first heterogeneous microparticles is less than or equal to 0.5 μm.
   Clause 23. The method of any clause or example herein, in particular, any one of Clauses 1-22, wherein:
for one or more of the first cores, the corresponding first payload includes a lubricant;
the providing of (a) comprises disposing the plurality of first heterogeneous microparticles on or within a structure; and
the subjecting of (b) is such that the lubricant released from the one or more first cores self-lubricates the structure.
   Clause 24. The method of any clause or example herein, in particular, Clause 23, wherein the first triggering event comprises friction.
   Clause 25. The method of any clause or example herein, in particular, any one of Clauses 1-22, wherein:
for one or more of the first cores, the corresponding first payload includes a drug;
the providing of (a) comprises disposing the plurality of first heterogeneous microparticles on or within a patient; and
the subjecting of (b) is such that the drug released from the one or more first cores is delivered to the patient.
   Clause 26. The method of any clause or example herein, in particular, Clause 25, wherein the first triggering event comprises ultrasound, electromagnetic radiation, heat, or any combination of the foregoing applied to the patient.
   Clause 27. The method of any clause or example herein, in particular, any one of Clauses 25-26, wherein the first triggering event comprises pH, temperature, ionic concentration, or any combination of the foregoing on or within the patient.
   Clause 28. The method of any clause or example herein, in particular, any one of Clauses 25-27, wherein the first triggering event comprises in situ exposure to biology of the patient.
   Clause 29. The method of any clause or example herein, in particular, any one of Clauses 25-28, wherein at least one of the first shells comprises a ligand for bonding to a target tissue within the patient, and the providing of (a) is such that at least one of the first heterogeneous microparticles disposed within the patient bonds to the target tissue via said ligand.
   Clause 30. The method of any clause or example herein, in particular, Clause 29, wherein the subjecting of (b) is such that the drug released from the at least one of the first cores is delivered to the target tissue, surrounding tissue, or both.
   Clause 31. The method of any clause or example herein, in particular, any one of Clauses 25-30, wherein the drug comprises a radiopharmaceutical.
   Clause 32. The method of any clause or example herein, in particular, any one of Clauses 1-31, wherein, for at least one of the first heterogeneous microparticles:
the first shell further defines one or more additional cores partitioned from the first core and one or more additional orifices in fluid communication with respective ones of the additional cores;
one or more additional payloads are respectively disposed within the one or more additional cores; and
the first cap comprises multiple sub-portions, each sub-portion sealing a corresponding orifice of the first shell.
   Clause 33. The method of any clause or example herein, in particular, Clause 32, wherein, for at least one of the first heterogeneous microparticles, the subjecting of (b) is such that the first triggering event releases the one or more additional payloads from the one or more additional cores at a same time as releasing the first payload from the first core.
   Clause 34. The method of any clause or example herein, in particular, any one of Clauses 32-33, wherein, for at least one of the first heterogeneous microparticles, the subjecting of (b) is such that the first payload released from the first core combines or reacts with the one or more additional payloads released from the one or more additional cores.
   Clause 35. The method of any clause or example herein, in particular, Clause 34, wherein the first payload and the one or more additional payloads react to form an epoxy, an adhesive, a paint, a coating, a foam, or any combination of the foregoing.
   Clause 36. The method of any clause or example herein, in particular, any one of Clauses 34-35, wherein:
at least one of the first heterogeneous microparticles has epoxy-forming components separately disposed as respective payloads in the first core and in the one or more additional cores;
the providing of (a) comprises disposing the plurality of first heterogeneous microparticles on or within a structure; and
the subjecting of (b) is such that the epoxy-forming components released from the first core and the one or more additional cores mix to form an epoxy that seals the crack or damage.
   Clause 37. The method of any clause or example herein, in particular, Clause 36, wherein the first triggering event comprises a crack or damage to the structure.
   Clause 38. The method of any clause or example herein, in particular, any one of Clauses 34-35, wherein:
at least one of the first heterogeneous microparticles has epoxy-forming components separately disposed as respective payloads in the first core and in the one or more additional cores;
the providing of (a) comprises disposing the plurality of first heterogeneous microparticles on or within a polymeric separator of a battery; and
the subjecting of (b) is such that the epoxy-forming components released from the first core and the one or more additional cores mix to form an epoxy that seals the separator.
   Clause 39. The method of any clause or example herein, in particular, Clause 38, wherein the first triggering event comprises heat, charge flow through the separator, or any combination of the foregoing.
   Clause 40. The method of any clause or example herein, in particular, any one of Clauses 34-35, wherein:
at least one of the first heterogeneous microparticles has foam-forming components separately disposed as respective payloads in the first core and in the one or more additional cores;
the providing of (a) comprises disposing the plurality of first heterogeneous microparticles on or within a structure; and
the subjecting of (b) is such that the foam-forming components released from the first core and the one or more additional cores mix to form a fire-protection foam.
   Clause 41. The method of any clause or example herein, in particular, Clause 40, wherein the first triggering event comprises heat.
   Clause 42. The method of any clause or example herein, in particular, any one of Clauses 34-35, wherein:
at least one of the first heterogeneous microparticles has foam-forming components separately disposed as respective payloads in the first core and in the one or more additional cores;
the providing of (a) comprises disposing the plurality of first heterogeneous microparticles on or within a battery; and
the subjecting of (b) is such that the foam-forming components released from the first core and the one or more additional cores mix to form a fire-protection foam within or around the battery.
   Clause 43. The method of any clause or example herein, in particular, Clause 42, wherein the first triggering event comprises heat, current through the battery, or any combination of the foregoing.
   Clause 44. The method of any clause or example herein, in particular, any one of Clauses 32-43, wherein, for at least one of the first heterogeneous microparticles, the subjecting of (b) is such that the first triggering event initiates release of the one or more additional payloads from the one or more additional cores at a time after initiation of release of the first payload from the first core.
   Clause 45. The method of any clause or example herein, in particular, any one of Clauses 32-44, wherein, for at least one of the first heterogeneous microparticles, the first payload includes a first drug, and one of the additional payloads includes a second drug different from the first drug.
   Clause 46. The method of any clause or example herein, in particular, any one of Clauses 32-45, further comprising:
(c) providing a plurality of second heterogeneous microparticles, each second microparticle having a maximum cross-sectional dimension of less than or equal to 300 μm, each second microparticle comprising:
   a second shell defining a second core and a second orifice in fluid communication with the second core;
   a second payload disposed within the second core; and
   a second cap coupled to the second shell so as to seal the second orifice, each of
   the second cap and the second shell comprising a respective cured photomaterial.
      Clause 47. The method of any clause or example herein, in particular, Clause 46, wherein the providing of (a) and (c) are such that the shells of the first and second heterogeneous microparticles are formed in a common batch by direct laser writing.
      Clause 48. The method of any clause or example herein, in particular, any one of Clauses 46-47, wherein a chemical composition of the second payload of at least one of the second heterogeneous microparticles is different than a chemical composition of the first payload of at least one of the first heterogeneous microparticles.
      Clause 49. The method of any clause or example herein, in particular, any one of Clauses 46-48, wherein the subjecting of (b) comprises simultaneously subjecting the plurality of first heterogeneous microparticles and the plurality of second heterogeneous microparticles to the first triggering event, such that:
at least a portion of the first orifice of at least one of the first heterogeneous microparticles is exposed from the respective first cap so as to release, through the exposed first orifice portion, the corresponding first payload from the first core of the first shell; and
at least a portion of the second orifice of at least one of the second heterogeneous microparticles is exposed from the respective second cap so as to release, through the exposed second orifice portion, the corresponding second payload from the second core of the second shell.
   Clause 50. The method of any clause or example herein, in particular, any one of Clauses 46-48, wherein the subjecting of (b) comprises simultaneously subjecting the plurality of first heterogeneous microparticles and the plurality of second heterogeneous microparticles to the first triggering event, such that:
at a first time, at least a portion of the first orifice of at least one of the first heterogeneous microparticles is exposed from the respective first cap so as to release, through the exposed first orifice portion, the corresponding first payload from the first core of the first shell; and
at a second time later than or overlapping with the first time, at least a portion of the second orifice of at least one of the second heterogeneous microparticles is exposed from the respective second cap so as to release, through the exposed second orifice portion, the corresponding second payload from the second core of the second shell.
   Clause 51. The method of any clause or example herein, in particular, any one of Clauses 46-50, wherein at least one of the first payloads includes a first drug, and at least one of the second payloads includes a second drug different from the first drug.
   Clause 52. The method of any clause or example herein, in particular, any one of Clauses 46-51, wherein at least one of the first payloads and at least one of the second payloads include a same drug.
   Clause 53. The method of any clause or example herein, in particular, any one of Clauses 46-52, wherein a release characteristic of at least one of the first caps in response to a corresponding triggering event is substantially the same as a release characteristic of at least one of the second caps in response to the corresponding triggering event.
   Clause 54. The method of any clause or example herein, in particular, any one of Clauses 46-53, wherein a release characteristic of at least one of the first caps in response to a corresponding triggering event is different than a release characteristic of at least one of the second caps in response to a corresponding triggering event.
   Clause 55. The method of any clause or example herein, in particular, any one of Clauses 46-54, wherein the subjecting of (b) comprises simultaneously subjecting the plurality of first heterogeneous microparticles and the plurality of second heterogeneous microparticles to the first triggering event, such that:
at least a portion of the first orifice of at least one of the first heterogeneous microparticles is exposed from the respective first cap so as to release, through the exposed first orifice portion, the corresponding first payload from the first core of the first shell; and
each second orifice remains sealed by the respective second cap so as to retain the second payload within the second core of the second shell.
   Clause 56. The method of any clause or example herein, in particular, Clause 55, further comprising:
(d) subjecting the plurality of first heterogeneous microparticles and the plurality of second heterogeneous microparticles to a second triggering event, such that at least a portion of the second orifice of at least one of the heterogeneous microparticles is exposed from the respective second cap so as to release, through the exposed second orifice portion, the corresponding second payload from the second core of the second shell.
   Clause 57. The method of any clause or example herein, in particular, any one of Clauses 46-56, wherein a chemical composition of the second payload of at least one of the second heterogeneous microparticles is substantially the same as a chemical composition of the first payload of at least one of the first heterogeneous microparticles.

Clause 58. The method of any clause or example herein, in particular, any one of Clauses 1-57, wherein the first triggering event causes gradual degradation of each first cap over time.

Clause 59. The method of any clause or example herein, in particular, any one of Clauses 1-58, wherein the providing of (a) comprises forming the plurality of first heterogeneous microparticles by:
(a1) providing a first curable photomaterial;
(a2) forming at least part of each first shell by patterning the first curable photomaterial using electromagnetic radiation and/or a particle beam;
(a3) loading one or more liquids into the first cores of the first shells to form the first payloads;
(a4) providing a second curable photomaterial over the first shells; and
(a5) forming at least part of each first cap by patterning the second curable photomaterial using electromagnetic radiation and/or a particle beam.
   Clause 60. The method of any clause or example herein, in particular, any one of Clauses 1-58, wherein the providing of (a) comprises forming the plurality of first heterogeneous microparticles by:
(a1) providing a first curable photomaterial;
(a2) forming at least part of each first shell by direct laser writing in the first curable photomaterial;
(a3) loading one or more liquids into the first cores of the first shells to form the first payloads;
(a4) providing a second curable photomaterial over the first shells; and
(a5) forming at least part of each first cap by direct laser writing of the second curable photomaterial.
   Clause 61. The method of any clause or example herein, in particular, any one of Clauses 59-60, wherein at least the loading, the providing the second curable photomaterial, and the forming the first caps are performed with the first shells within a common microfluidic channel.
   Clause 62. The method of any clause or example herein, in particular, any one of Clauses 59-61, wherein the loading of (a3) comprises at least one of: (i) creating a pressure less than atmospheric pressure (e.g., a vacuum) within the first cores; (ii) diffusing the one or more liquids into the first cores; (iii) applying positive pressure to the one or more liquids; or (iv) any combination of the foregoing.
   Clause 63. The method of any clause or example herein, in particular, any one of Clauses 59-62, wherein the patterning of (a2) or (a5) comprises inducing two-photon polymerization within the respective curable photomaterial by focused illumination from a femtosecond infrared laser.
   Clause 64. The method of any clause or example herein, in particular, any one of Clauses 1-63, wherein the providing of (a) comprises preloading the plurality of first heterogeneous microparticles onto or within a structure.
   Clause 65. The method of any clause or example herein, in particular, Clause 64, wherein the preloading comprises:
printing, spraying, or coating the plurality of first heterogeneous microparticles onto the structure;
infiltrating, infusing, injecting, or impregnating the plurality of first heterogeneous microparticles into the structure;
embedding the plurality of first heterogeneous microparticles within the structure; or
any combination of the foregoing.
   Clause 66. The method of any clause or example herein, in particular, any one of Clauses 1-63, wherein the providing of (a) comprises:
infusing the plurality of first heterogeneous microparticles into a patient;
injecting the plurality of first heterogeneous microparticles into a patient;
ingesting of the plurality of first heterogeneous microparticles by the patient; or
any combination of the foregoing.
   Clause 67. The method of any clause or example herein, in particular, any one of Clauses 1-66, wherein, for each of the first heterogeneous microparticles:
a wall thickness of the first shell is less than or equal to 20% of the maximum cross-sectional dimension;
a diameter of the first orifice is less than or equal to 50% of the maximum cross-sectional dimension;
a height of the first cap along a radial direction of the corresponding first shell is less than or equal to 20% of the maximum cross-sectional dimension; or
any combination of the foregoing.
   Clause 68. The method of any clause or example herein, in particular, any one of Clauses 1-67, wherein the maximum cross-sectional dimension is 200 μm or less, 100 μm or less, 50 μm or less, 25 μm or less, or 10 μm or less.
   Clause 69. A heterogeneous microparticle comprising:
a shell defining a first core and a first orifice in fluid communication with the first core;
a first payload disposed within the first core; and
a cap coupled to the shell so as to seal the first orifice,
wherein the cap and the shell each comprise a respective cured photomaterial.
   Clause 70. The heterogeneous microparticle of any clause or example herein, in particular, Clause 69, wherein a maximum cross-sectional dimension of the microparticle is less than or equal to 300 μm.
   Clause 71. The heterogeneous microparticle of any clause or example herein, in particular, any one of Clauses 69-70, wherein a maximum cross-sectional dimension of the microparticle is 200 μm or less, 100 μm or less, 50 μm or less, 25 μm or less, or 10 μm or less.
   Clause 72. The heterogeneous microparticle of any clause or example herein, in particular, any one of Clauses 69-71, wherein a material composition of the shell is different than a material composition of the cap, a material composition of a first part of the shell is different than a material composition of a second part of the shell, a material composition of a first part of the cap is different than a material composition of a second part of the cap, or any combination of the foregoing.
   Clause 73. The heterogeneous microparticle of any clause or example herein, in particular, any one of Clauses 69-72, wherein the shell is formed of a biocompatible material and the cap is formed of a biodegradable or biocompatible material.
   Clause 74. The heterogeneous microparticle of any clause or example herein, in particular, any one of Clauses 69-73, wherein both the shell and the cap are formed of biodegradable materials.
   Clause 75. The heterogeneous microparticle of any clause or example herein, in particular, any one of Clauses 69-74, where the cap is constructed to degrade faster in response to a triggering event than the shell.
   Clause 76. The heterogeneous microparticle of any clause or example herein, in particular, any one of Clauses 69-75, wherein the first payload comprises at least one liquid phase component within the core.
   Clause 77. The heterogeneous microparticle of any clause or example herein, in particular, any one of Clauses 69-76, wherein the first payload comprises:
a medicament, a nutrient, a repellent, a pesticide, a protectant, an adhesive, a deterrent, a fragrance, a cosmetic, a lubricant, or a component for forming any of the foregoing;
a living cellular organism or virus;
a powder; or
any combination of the above.
   Clause 78. The heterogeneous microparticle of any clause or example herein, in particular, any one of Clauses 69-77, wherein the cap is constructed to be partially or fully removed to expose at least a portion of the first orifice when subjected to a triggering event.
   Clause 79. The heterogeneous microparticle of any clause or example herein, in particular, Clause 78, wherein the cap is constructed to be partially or fully removed when exposed to one or more environmental conditions as the triggering event.
   Clause 80. The heterogeneous microparticle of any clause or example herein, in particular, Clause 79, wherein the one or more environmental conditions comprise stress, strain, pressure, fracture, friction, wear, electromagnetic radiation, temperature, sensible heat, latent heat, pH, ionic concentration, chemical, biological environment, or any combination of the foregoing.
   Clause 81. The heterogeneous microparticle of any clause or example herein, in particular, any one of Clauses 78-80, wherein the cap is constructed to be partially or fully removed when energy or force is applied as the triggering event.
   Clause 82. The heterogeneous microparticle of any clause or example herein, in particular, Clause 80, wherein the energy or force comprises electromagnetic radiation, electrical energy, magnetic energy, vibrational energy, ultrasonic energy, sensible heat, latent heat, or any combination of the foregoing.
   Clause 83. The heterogeneous microparticle of any clause or example herein, in particular, any one of Clauses 78-82, wherein the cap is constructed to degrade over time when subjected to the triggering event.
   Clause 84. The heterogeneous microparticle of any clause or example herein, in particular, any one of Clauses 69-83, wherein the cap is patterned such that, in response to the first triggering event, an area of the first orifice exposed from the cap changes over time so as to regulate release of the first payload from the first core.
   Clause 85. The heterogeneous microparticle of any clause or example herein, in particular, Clause 84, wherein the cap has a geometric pattern or a fractal pattern.
   Clause 86. The heterogeneous microparticle of any clause or example herein, in particular, any one of Clauses 84-85, wherein a feature size of the pattern in the cap is less than or equal to 100 nm.
   Clause 87. The heterogeneous microparticle of any clause or example herein, in particular, any one of Clauses 69-86, wherein:
the shell further defines one or more additional cores partitioned from the first core and one or more additional orifices in fluid communication with respective ones of the additional cores;
the heterogeneous microparticle further comprises one or more additional payloads respectively disposed within the one or more additional cores; and
the cap comprises multiple sub-portions, each sub-portion sealing a corresponding orifice of the shell.
   Clause 88. The heterogeneous microparticle of any clause or example herein, in particular, Clause 87, wherein the first payload and the one or more additional payloads comprise separate components or reactants for subsequent combination when released from the shell.
   Clause 89. The heterogeneous microparticle of any clause or example herein, in particular, any one of Clauses 87-88, wherein the first payload and the one or more additional payloads comprise separate components or reactants for forming an epoxy, an adhesive, a paint, a coating, a foam, or any combination of the foregoing.
   Clause 90. The heterogeneous microparticle of any clause or example herein, in particular, any one of Clauses 87-88, wherein the first payload includes a first drug, and one of the additional payloads includes a second drug different from the first drug.
   Clause 91. The heterogeneous microparticle of any clause or example herein, in particular, any one of Clauses 87-87, wherein the first payload includes a first drug at a first concentration, and one of the additional payloads includes the first drug at a second concentration different from the first concentration.
   Clause 92. The heterogeneous microparticle of any clause or example herein, in particular, any one of Clauses 87-91, wherein:
a sub-portion of the cap sealing the first orifice is constructed such that, in response to a triggering event, at least a portion of the first orifice is exposed to release the first payload at a first time; and
a sub-portion of the cap sealing one of the additional orifices is constructed such that, in response to the triggering event, at least a portion of the one of the additional orifices is exposed to release one of the additional payloads at a second time different than the first time.
   Clause 93. A system comprising a plurality of the heterogeneous microparticle, each microparticle according to any clause or example herein, in particular, any one of Clauses 69-92.
   Clause 94. The system of any clause or example herein, in particular, Clause 93, wherein a standard deviation of the maximum cross-sectional dimensions within the plurality of the heterogeneous microparticles is less than or equal to 5% of an average of the maximum cross-sectional dimensions within the plurality of the heterogeneous microparticles, or less than or equal to 2% of an average of the maximum cross-sectional dimensions within the plurality of the heterogeneous microparticles.
   Clause 95. The system of any clause or example herein, in particular, any one of Clauses 93-94, wherein a standard deviation of the maximum cross-sectional dimensions within the plurality of heterogeneous microparticles is less than or equal to 0.5 μm.
   Clause 96. The system of any clause or example herein, in particular, any one of Clauses 93-95, further comprising a structure, wherein the plurality of heterogeneous microparticles are disposed on or within the structure.
   Clause 97. The system of any clause or example herein, in particular, Clause 96, wherein the structure comprises a battery or a component thereof.
   Clause 98. The system of any clause or example herein, in particular, Clause 96, wherein the structure comprises vehicle, building, casing, civil infrastructure, machinery, biological implant, furniture, or a component thereof.
   Clause 99. The system of any clause or example herein, in particular, Clause 96, wherein the structure comprises a biological implant or a component thereof.
   Clause 100. The system of any clause or example herein, in particular, any one of Clauses 93-99, further comprising a control unit, wherein the control unit comprises one or more processors and computer readable storage media.
   Clause 101. The system of any clause or example herein, in particular, Clause 100, wherein the computer readable storage media stores instructions that, when executed by the one or more processors, cause the one or more processors to perform or to issue control signals to cause performance of one or more aspects of the method of any clause or example herein, in particular, any one of Clauses 1-68.
   Clause 102. The system of any clause or example herein, in particular, any one of Clauses 100-101, further comprising a device constructed to apply to the microparticles at least one of electromagnetic radiation (e.g., a visible light source, an X-ray source, an infrared light source, an ultraviolet light source, etc.), electrical energy (e.g., a current source, a voltage source, etc.), magnetic energy (e.g., an electromagnet), vibrational energy (e.g., a shaker, acoustic transducer, etc.), ultrasonic energy (e.g., an ultrasound transducer), sensible or latent heat (e.g., via conductive, convective, and/or radiative heating), or any combination of the foregoing.

### Conclusion

Any of the features illustrated or described herein, for example, with respect to FIGS. 1A-9H and Clauses 1-102, can be combined with any other feature illustrated or described herein, for example, with respect to FIGS. 1A-9H and Clauses 1-102 to provide materials, structures, methods, and embodiments not otherwise illustrated or specifically described herein. For example, the cap design 152 of FIGS. 1E-1F (or any other cap design for controlling release of microparticle payload) can be applied to any of the caps of FIGS. 1A-1D, 2A-2H, 4D-4F, 5B-6E, and 7C-7D. All features described herein are independent of one another and, except where structurally impossible, can be used in combination with any other feature described herein.

In view of the many possible embodiments to which the principles of the disclosed technology may be applied, it should be recognized that the illustrated embodiments are only examples and should not be taken as limiting the scope of the disclosed technology. Rather, the scope is defined by the following claims. We therefore claim all that comes within the scope and spirit of these claims.

1. A method comprising:
(a) providing a plurality of first heterogeneous microparticles, each first heterogeneous microparticle having a maximum cross-sectional dimension of less than or equal to 300 μm, each first heterogeneous microparticle comprising:
a first shell defining a first core and a first orifice in fluid communication with the first core;
a first payload disposed within the first core; and
a first cap coupled to the first shell so as to seal the first orifice, the first cap and the first shell each comprising a respective cured photomaterial; and
(b) subjecting the plurality of first heterogeneous microparticles to a first triggering event, such that at least a portion of each first orifice is exposed from the respective first cap so as to release, through the exposed first orifice portion, the corresponding first payload from the first core of the first shell.

2-5. (canceled)

6. The method of claim 1, wherein one or more of the first payloads comprises at least one liquid phase component within the respective first core.

7. (canceled)

8. The method of claim 1, wherein one or more of the first payloads comprises a living cellular organism or virus.

9-10. (canceled)

11. The method of claim 1, wherein:
one or more of the first payloads comprises a powder, and
the providing of (a) comprises forming the powder in situ within the corresponding first core by freeze drying.

12. (canceled)

13. The method of claim 1, wherein:
the first triggering event comprises exposure to one or more environmental conditions, and
the one or more environmental conditions comprise stress, strain, pressure, fracture, friction, wear, electromagnetic radiation, temperature, sensible heat, latent heat, pH, ionic concentration, chemical, biological environment, or any combination of the foregoing.

14. (canceled)

15. The method of claim 1, wherein:
the first triggering event comprises active application of energy or force to the plurality of first heterogeneous microparticles, and
the energy or force comprises electromagnetic radiation, electrical energy, magnetic energy, vibrational energy, ultrasonic energy, sensible heat, latent heat, or any combination of the foregoing.

16. The method of claim 1, wherein, for one or more of the first heterogeneous microparticles, the first cap is patterned such that, in response to the first triggering event, an area of the first orifice exposed from the first cap changes over time so as to regulate release of the first payload from the first core.

17. The method of claim 16, wherein one or more of the first payloads includes a drug.

18-22. (canceled)

23. The method of claim 1, wherein:
for one or more of the first cores, the corresponding first payload includes a lubricant;
the providing of (a) comprises disposing the plurality of first heterogeneous microparticles on or within a structure; and
the subjecting of (b) is such that the lubricant released from the one or more first cores self-lubricates the structure.

24. The method of claim 23, wherein the first triggering event comprises friction.

25. The method of claim 1, wherein:
for one or more of the first cores, the corresponding first payload includes a drug;
the providing of (a) comprises disposing the plurality of first heterogeneous microparticles on or within a patient; and
the subjecting of (b) is such that the drug released from the one or more first cores is delivered to the patient.

26. The method of claim 25, wherein:
(i) the first triggering event comprises ultrasound, electromagnetic radiation, heat, or any combination of the foregoing applied to the patient;
(ii) the first triggering event comprises pH, temperature, ionic concentration, or any combination of the foregoing on or within the patient; or
(iii) the first triggering event comprises in situ exposure to biology of the patient.

27-28. (canceled)

29. The method of claim 25, wherein at least one of the first shells comprises a ligand for bonding to a target tissue within the patient, and the providing of (a) is such that at least one of the first heterogeneous microparticles disposed within the patient bonds to the target tissue via said ligand.

30-31. (canceled)

32. The method of claim 1, wherein, for at least one of the first heterogeneous microparticles:
the first shell further defines one or more additional cores partitioned from the first core and one or more additional orifices in fluid communication with respective ones of the additional cores;
one or more additional payloads are respectively disposed within the one or more additional cores; and
the first cap comprises multiple sub-portions, each sub-portion sealing a corresponding orifice of the first shell.

33-45. (canceled)

46. The method of claim 32, further comprising:
(c) providing a plurality of second heterogeneous microparticles, each second microparticle having a maximum cross-sectional dimension of less than or equal to 300 μm, each second microparticle comprising:
a second shell defining a second core and a second orifice in fluid communication with the second core;
a second payload disposed within the second core; and
a second cap coupled to the second shell so as to seal the second orifice, each of the second cap and the second shell comprising a respective cured photomaterial.

47-58. (canceled)

59. A method comprising:
(a1) providing a first curable photomaterial;
(a2) forming at least part of first shells of first heterogeneous microparticles by patterning the first curable photomaterial using electromagnetic radiation or a particle beam, each first shell defining a first core and a first orifice in fluid communication with the first core;
(a3) loading one or more liquids into the first cores of the first shells to form first payloads;
(a4) providing a second curable photomaterial over the first shells; and
(a5) forming at least part of first caps of the first heterogenous microparticles by patterning the second curable photomaterial using electromagnetic radiation or a particle beam,
wherein each first heterogeneous microparticle has a maximum cross-sectional dimension of less than or equal to 300 μm.

60. The method of claim 59, wherein at least the loading, the providing the second curable photomaterial, and the forming the first caps are performed with the first shells within a common microfluidic channel.

61-66. (canceled)

67. A heterogeneous microparticle comprising:
a shell defining a first core and a first orifice in fluid communication with the first core;
a first payload disposed within the first core; and
a cap coupled to the shell so as to seal the first orifice,
wherein the cap and the shell each comprise a respective cured photomaterial.

68-74. (canceled)

75. The heterogeneous microparticle of claim 67, wherein the first payload comprises:
a medicament, a nutrient, a repellent, a pesticide, a protectant, an adhesive, a deterrent, a fragrance, a cosmetic, a lubricant, or a component for forming any of the foregoing;
a living cellular organism or virus;
a powder; or
any combination of the above.

76-81. (canceled)

82. The heterogeneous microparticle of claim 67, wherein the cap is patterned such that, in response to a triggering event, an area of the first orifice exposed from the cap changes over time so as to regulate release of the first payload from the first core.

83-97. (canceled)
